(19) United States
(12) Patent Application Publication
(10) Pub. No.: US 2026/0020804 A1
(43) Pub. Date: Jan. 22, 2026

(54) MANUFACTURING METHOD OF SELF-HEALING ADHESIVE PATCH FOR ELECTRONIC SKIN SENSOR AND SELF-HEALING ADHESIVE PATCH FOR ELECTRONIC SKIN SENSOR MANUFACTURED THROUGH THIS METHOD

(71) Applicants: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(72) Inventors: Jin Young OH, Anyang-si (KR); Chang Hyun PANG, Suwon-si (KR); Min Woo JEONG, Gwangmyeong-si (KR); Da Wan KIM, Suwon-si (KR); Do Hyun LIM, Suwon-si (KR)

(73) Assignees: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(21) Appl. No.: 19/212,008

(22) Filed: May 19, 2025

(30) Foreign Application Priority Data
Jul. 22, 2024 (KR) 10-2024-0096252

(51) Int. Cl. A61B 5/257; A61B 5/00

(52) U.S. Cl. CPC A61B 5/257; A61B 5/6833; A61B 2560/0468; A61B 2562/125

(57) ABSTRACT

The present invention discloses a method for manufacturing a self-healing adhesive patch for electronic skin sensors and a self-healing adhesive patch for electronic skin sensors manufactured thereby. The present invention is characterized in that the self-healing adhesive patch comprises: a first elastomer film layer including a polymer compound and an isocyanate-based compound, a second elastomer film layer formed on the first elastomer film layer and including a polymer compound and the isocyanate-based compound, and an electrode formed on the second elastomer film layer.

### CROSS-REFERENCE TO RELATED APPLICATION

This application claims priority from and the benefit of Korean Patent Application No. 10-2024-0096252 filed on Jul. 22, 2024, in the Korean Intellectual Property Office, the disclosure of which is incorporated herein by reference.

### BACKGROUND OF THE DISCLOSURE

### Field of the Disclosure

Example embodiments relate to a method of manufacturing a self-healing adhesive patch for an electronic skin sensor, in which a multilayer structure is used to fabricate the patch, thereby enhancing mechanical properties, self-healing characteristics, and adhesive performance, and to a self-healing adhesive patch for an electronic skin sensor manufactured thereby.

### Description of the Related Art

With advances in flexible conductive polymers and hybrid materials, stretchable electronic devices such as bioelectronics or electronic skin (E-skin), which can be attached to biological organs, have recently garnered significant attention as next-generation technologies in biomedical and robotic applications. In addition to various mechanical and electrical properties such as elasticity, adhesiveness, and conductivity, increasing importance is being placed on the development of devices capable of natural self-healing from physical wear and damage, especially when applied to biological systems.

Self-healing materials have been developed to address mechanical damage and restore sensing functions, with the goal of improving the durability and operational lifetime of electronic devices. Such approaches not only extend the overall service life of electronic products but also contribute to reducing electronic waste.

To develop robust bioinspired electronic devices, it is essential to employ stretchable and self-healing materials that possess both softness and resilience against mechanical damage. These materials must exhibit excellent resistance to damage, prevent the propagation of microcracks, and possess healing and recovery capabilities when cracks occur-all of which are essential prerequisites for constructing durable skin-integrated electronic systems.

Conventional intrinsically self-healable materials typically include soft rubber-like polymer components. While enhancing the strength or concentration of dynamic bonds within such polymers can improve tensile strength, it may also degrade tensile properties and dynamic behavior. Accordingly, recent studies have explored various approaches for developing self-healing polymers based on molecular structure control, combinations of strong and weak dynamic bonds, and incorporation of nano- and micro-structured architectures.

Despite advances in self-healing polymers (SHPs) with improved mechanical properties, challenges remain in achieving reversible adhesion and conformal contact with both dry and moist rough surfaces for bioelectronic interfaces designed for skin attachment. Self-healing adhesives based on ester-crosslinked polymers-such as chitosan, polyallylamine, and polyacrylic acid-have shown excellent adhesion to various skin electronic surfaces and demonstrate strong adhesion and compliance to skin and tissue. However, their long-term sustainability requires further investigation due to potential chemical contamination and issues with reusability.

For the practical application of self-healing polymers (SHPs), in addition to excellent adhesive performance and biocompatibility, the materials' mechanical properties often fall short compared to non-self-healing materials due to their composition based mainly on dynamic bonds and low glass transition temperatures. As a result, fabrication of microstructures using SHPs has proven difficult, especially in terms of patterning cracks, defects, and stretchability.

Furthermore, due to enhanced chain dynamics above the glass transition temperature, maintaining structural integrity over time has been a major challenge for conventional self-healing polymers. Therefore, the development of functional microstructured materials and interfaces that possess autonomous self-healing capability along with reversible adhesion, conductivity, and stretchability is expected to open new avenues for a wide range of applications. In this regard, the design of novel self-healing adhesive structures suitable for skin-adhesive electronic devices is of critical importance.

### SUMMARY

An object of the present invention is to provide a method of manufacturing a self-healing adhesive patch for an electronic skin sensor, in which an elastomer film layer is fabricated in a multilayer structure to enhance mechanical properties, self-healing characteristics, and adhesive performance, and to provide a self-healing adhesive patch for an electronic skin sensor manufactured thereby.

Another object of the present invention is to provide a method of manufacturing a self-healing adhesive patch for an electronic skin sensor, which can be applied as a biosensor and maintain its original properties under various environmental conditions, body movements, and even mechanical cuts, thereby enabling stable and reliable measurement of biosignals, and to provide a self-healing adhesive patch for an electronic skin sensor manufactured thereby.

According to one embodiment of the present invention, a self-healing adhesive patch for an electronic skin sensor includes:
a first elastomer film layer comprising a polymer compound and an isocyanate-based compound, a second elastomer film layer formed on the first elastomer film layer, the second elastomer film layer comprising the polymer compound and the isocyanate-based compound, and an electrode formed on the second elastomer film layer.

The isocyanate-based compound comprises a first isocyanate-based compound and a second isocyanate-based compound, and a first molar ratio of the first isocyanate-based compound to the second isocyanate-based compound in the first elastomer film layer is different from a second molar ratio of the first isocyanate-based compound to the second isocyanate-based compound in the second elastomer film layer.

The first and second elastomer film layers may be configured to control their respective Young's moduli by adjusting hydrogen bonding between the polymer compound and the isocyanate-based compound.

The first molar ratio and the second molar ratio may each be in a range of 0.1:0.9 to 0.9:0.1.

A thickness ratio of the first elastomer film layer to the second elastomer film layer may be in a range of 1:2 to 2:1.

The second elastomer film layer may comprise a pattern.

The self-healing adhesive patch may further comprise a third elastomer film layer formed on the second elastomer film layer, the third elastomer film layer comprising the polymer compound and the isocyanate-based compound.

The isocyanate-based compound may include the first isocyanate-based compound and the second isocyanate-based compound.

A third molar ratio of the first isocyanate-based compound to the second isocyanate-based compound in the third elastomer film layer may be different from the first molar ratio and the second molar ratio.

The third molar ratio may be in a range of 0.1:0.9 to 0.9:0.1.

A thickness of the third elastomer film layer may be in a range of 20 μm to 30 μm.

The polymer compound may comprise at least one selected from the group consisting of polydimethylsiloxane (PDMS), polyethylene oxide (PEO), perfluoropolyether (PFPE), polybutylene (PB), poly (ethylene-co-1-butylene), poly (butadiene), hydrogenated poly (butadiene), poly (ethylene oxide)-poly (propylene oxide) block copolymer, and poly (hydroxyalkanoate).

The isocyanate-based compound may comprise at least one selected from the group consisting of 4,4′-methylenebis (phenyl isocyanate) (MPI), isophorone diisocyanate (IPDI), cyclohexylene diisocyanate, hydrogenated toluene diisocyanate (hydrogenated TDI), bis(2-isocyanatoethyl)-4-diclohexene-1,2-dicarboxylate, 2,5-norbornene diisocyanate, and 2,6-norbornene diisocyanate.

The method of manufacturing the self-healing adhesive patch may comprise:
forming the second elastomer film layer on the first elastomer film layer to produce an elastomer patch;
patterning the elastomer patch to produce an elastic adhesive patch;
performing plasma surface treatment on the elastic adhesive patch to produce a surface-modified elastic adhesive patch; and
depositing an electrode on the surface-modified elastic adhesive patch to produce the self-healing adhesive patch.

In the step of producing the elastomer patch, the isocyanate-based compound comprises the first isocyanate-based compound and the second isocyanate-based compound, and the first molar ratio of the first isocyanate-based compound to the second isocyanate-based compound in the first elastomer film layer may be different from the second molar ratio in the second elastomer film layer.

The first and second molar ratios may each be in a range of 0.1:0.9 to 0.9:0.1.

In the step of producing the elastomer patch, the method may further comprise forming a third elastomer film layer on the second elastomer film layer, wherein the third elastomer film layer comprises the polymer compound and the isocyanate-based compound, the isocyanate-based compound comprises the first isocyanate-based compound and the second isocyanate-based compound, and a third molar ratio of the first isocyanate-based compound to the second isocyanate-based compound in the third elastomer film layer is different from the first molar ratio and the second molar ratio.

The third molar ratio may be in a range of 0.1:0.9 to 0.9:0.1.

In the step of producing the elastic adhesive patch, the patterning may be performed using one method selected from the group consisting of imprinting, 3D printing, and contact printing.

According to one embodiment of the present invention, there is provided a method of manufacturing a self-healing adhesive patch for an electronic skin sensor, and a self-healing adhesive patch manufactured thereby, which is intrinsically self-healable by using an elastomer film layer comprising a polymer compound and an isocyanate-based compound, and which offers excellent healing and recovery capabilities against mechanical damage.

According to another embodiment of the present invention, by laminating elastomer film layers comprising controlled molar ratios of a polymer compound and an isocyanate-based compound into a multilayer structure, it is possible to provide a method of manufacturing a self-healing adhesive patch for an electronic skin sensor, and a self-healing adhesive patch manufactured thereby, in which improved mechanical properties, self-healing characteristics, and adhesive performance are achieved.

According to still another embodiment of the present invention, by applying patterning and electrode deposition on the adhesive surface, it is possible to provide a method of manufacturing a self-healing adhesive patch for an electronic skin sensor, and a self-healing adhesive patch manufactured thereby, which not only maintains excellent pattern integrity and self-healing properties but also enables stable and reliable biosignal monitoring under various environmental conditions, body movements, and even mechanical cuts when applied as a biosensor.

### BRIEF DESCRIPTION OF THE FIGURES

Embodiments will be described in more detail with regard to the figures, wherein like reference numerals refer to like parts throughout the various figures unless otherwise specified, and wherein:

FIG. 1 is a schematic view of a self-healing adhesive patch for an electronic skin sensor including a double-layered elastomer film layer according to one embodiment of the present invention.

FIG. 2 is a schematic view of a self-healing adhesive patch for an electronic skin sensor including a triple-layered elastomer film layer according to one embodiment of the present invention.

FIGS. 3 and 4 are schematic views illustrating a self-healing mechanism of the self-healing adhesive patch for an electronic skin sensor according to one embodiment of the present invention.

FIG. 5 is a flowchart illustrating a method of manufacturing the self-healing adhesive patch for an electronic skin sensor according to one embodiment of the present invention.

FIG. 6 is a graph showing the results of 1H nuclear magnetic resonance (NMR) spectroscopy depending on the molar ratio of MPU and IU.

FIG. 7 is a graph showing small-angle X-ray scattering (SAXS) analysis depending on the molar ratio of MPU and IU.

FIGS. 8 to 10 are graphs showing differential scanning calorimetry (DSC) analysis depending on the molar ratio of MPU and IU.

FIG. 11 is a graph showing Young's modulus depending on the molar ratio of MPU and IU.

FIG. 12 is a graph evaluating rheological properties depending on the molar ratio of MPU and IU.

FIG. 13 is a graph showing flow transition relaxation time (Tf) and segmental relaxation time (Ts) depending on the molar ratio of MPU and IU.

FIG. 14 is a graph comparing activation energy (Eₐ,flow) depending on the molar ratio of MPU and IU.

FIG. 15 is an image showing pattern retention over time at various MPU to IU molar ratios, viewed from side and top.

FIG. 16 is an image showing pattern reflow behavior based on the Laplace effect and interchain interactions.

FIGS. 17 and 18 are graphs evaluating structural stability over time at various MPU to IU molar ratios.

FIG. 19 is an image comparing structural stability at different MPU to IU molar ratios in Example 1-1.

FIG. 20 is a graph evaluating adhesive retention at different MPU to IU molar ratios in Example 1-1.

FIG. 21 is a graph comparing adhesive strength under various environmental conditions between Example 1-1 and Comparative Example 1.

FIG. 22 is an image showing the shape of the patch during attachment and detachment in Example 1.

FIG. 23 is an image comparing conformal contact between Example 1-2 and Comparative

### Example 1.

FIG. 24 is a schematic diagram and optical microscope (OM) image comparing conformal contact with and without PDMS-MPU_{0.2}-IU_{0.8}.

FIG. 25 is an OM image comparing conformal contact between Example 1-1 and Example 1-2.

FIG. 26 is a graph evaluating adhesive strength under various environmental conditions for different self-healing adhesive patches.

FIGS. 27 and 28 are graphs evaluating repeatable adhesion and recovery performance in

### Example 1-2.

FIG. 29 is an image evaluating self-adhesive healing performance in Example 1-2.

FIGS. 30 and 31 are graphs evaluating self-healing characteristics and mechanical properties according to the layer thickness ratio in Examples 1-1 and 1-2.

FIG. 32 is an image showing atomic force microscopy (AFM) analysis of the multilayer interface in Example 1-2.

FIG. 33 is a graph evaluating self-healing characteristics depending on the presence or absence of a pattern in Example 2.

FIG. 34 is an OM image showing the self-healing process of a fractured surface in Example 2.

FIG. 35 is a graph evaluating surface energy depending on oxygen (O₂) plasma treatment time in Example 3.

FIG. 36 is a graph evaluating electrical conductivity depending on the thickness of gold (Au) deposition in Example 3.

FIG. 37 is a graph evaluating adhesive strength under various environmental conditions after gold (Au) deposition in Example 3.

FIG. 38 is a graph evaluating electrical conductivity over self-healing time in Example 3.

FIG. 39 is a graph evaluating electrical conductivity under tensile deformation in Example 3.

FIG. 40 is an image comparing skin irritation between Example 3 and Comparative Example 2.

FIG. 41 is a graph comparing performance in underwater conditions between Example 3 and Comparative Example 2.

FIG. 42 is a graph evaluating real-time self-healing behavior in Example 3.

FIG. 43 is a graph evaluating dynamic motion in real time in Example 3.

FIG. 44 is an image evaluating reusability of the self-healing adhesive patch in Example 3.

### DETAILED DESCRIPTION OF THE DISCLOSURE

Hereinafter, exemplary embodiments of the present invention will be described in detail with reference to the accompanying drawings and the contents described therein. However, the present invention is not limited to or restricted by these embodiments.

The terminology used herein is for the purpose of describing particular embodiments only and is not intended to limit the present invention. As used herein, the singular forms “a” and “an” include plural referents unless the context clearly dictates otherwise. The terms “comprises” and “comprising” as used herein specify the presence of stated features, steps, operations, or components but do not preclude the presence or addition of one or more other features, steps, operations, or components.

Further, expressions such as “embodiment,” “example,” “aspect,” and “illustration” used herein are not intended to suggest that one particular feature or configuration is superior or advantageous over others.

Additionally, the term “or” as used herein is intended to mean inclusive “or” rather than exclusive “or,” unless otherwise stated or unless the context clearly indicates otherwise. That is, the phrase “A or B” is intended to encompass “A,” “B,” or both “A and B.”

Unless otherwise defined, all terms (including scientific and technical terms) used herein shall be interpreted as having the same meaning as commonly understood by one of ordinary skill in the art to which the invention pertains. Terms that are defined in general dictionaries are to be interpreted consistently with their meaning in the context of the relevant technical field and are not to be interpreted in an idealized or overly formal sense unless explicitly so defined herein.

In describing the present invention, specific descriptions of known configurations or functions may be omitted to avoid obscuring the gist of the invention. Also, the terminology used in the specification is intended to appropriately describe the embodiments of the invention, and the meaning of such terms should be interpreted based on the entire content of the specification. Some terms may have been arbitrarily selected by the applicant, in which case such terms will be clearly defined in the corresponding part of the description.

Despite the development of self-healing polymers (SHPs) with improved mechanical properties, such materials still face limitations in achieving reversible adhesion and conformal contact with rough, dry, or moist surfaces. While self-healing adhesives based on ester-crosslinked polymers have been developed to address this issue and offer high adhesion strength, they may still pose challenges such as potential chemical contamination, mechanical deficiencies, and difficulty in fabricating fine structures using SHPs due to cracking, defects, and elongation.

Accordingly, in one embodiment of the present invention, a self-healing adhesive patch for an electronic skin sensor may be provided by controlling the molar ratio of a polymer compound and an isocyanate-based compound in an elastomer film layer, and by laminating such elastomer film layers with controlled molar ratios into a multilayer structure. As a result, superior mechanical properties-including self-healing characteristics, adhesive performance, biocompatibility, electrical conductivity, and stretchability-can be achieved when compared to single-layer structures.

Furthermore, in another embodiment of the present invention, the self-healing adhesive patch (100, 200) for an electronic skin sensor can be applied as a stretchable electronic device such as a bioelectronic interface or electronic skin (E-skin) that is attachable to biological tissues or organs, and may exhibit excellent applicability in next-generation biomedical and robotic applications due to its high potential and versatility.

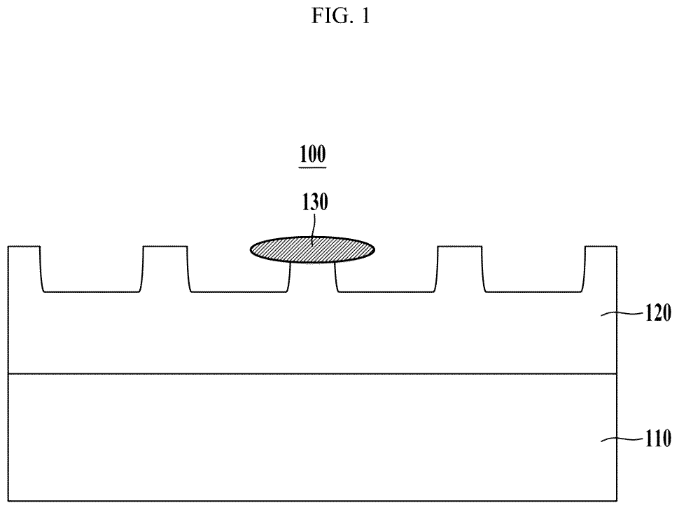

Hereinafter, a self-healing adhesive patch for an electronic skin sensor according to one embodiment of the present invention will be described with reference to FIGS. 1 to 4.

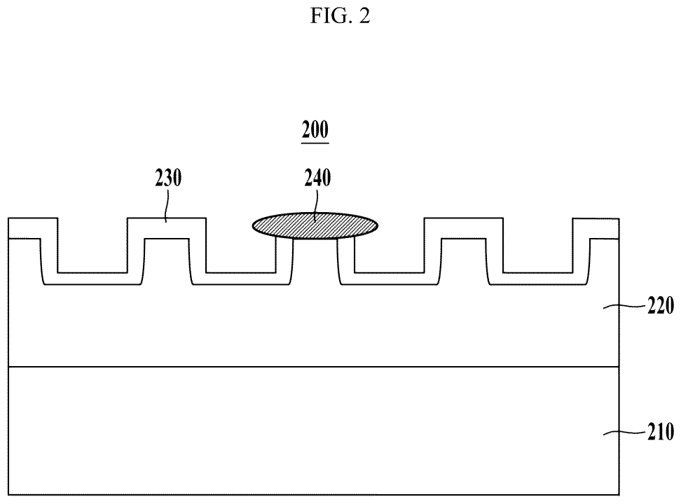

FIG. 1 is a schematic view of a self-healing adhesive patch for an electronic skin sensor including a double-layered elastomer film structure according to one embodiment of the present invention, and FIG. 2 is a schematic view of a self-healing adhesive patch for an electronic skin sensor including a triple-layered elastomer film structure according to one embodiment of the present invention.

Referring to FIG. 1, a self-healing adhesive patch 100, 200 for an electronic skin sensor according to one embodiment of the present invention comprises: a first elastomer film layer 110, 210 comprising a polymer compound and an isocyanate-based compound; a second elastomer film layer 120, 220 formed on the first elastomer film layer 110, 210, the second elastomer film layer comprising the polymer compound and the isocyanate-based compound; and an electrode 130, 240 formed on the second elastomer film layer 120, 220.

The isocyanate-based compound may comprise a first isocyanate-based compound and a second isocyanate-based compound.

In particular, in the self-healing adhesive patch 100, 200 according to one embodiment of the present invention, a first molar ratio of the first isocyanate-based compound to the second isocyanate-based compound in the first elastomer film layer 110, 210 may be different from a second molar ratio of the first isocyanate-based compound to the second isocyanate-based compound in the second elastomer film layer 120, 220.

Here, the “first molar ratio” refers to the molar ratio of the first and second isocyanate-based compounds included in the first elastomer film layer 110, 210, and the “second molar ratio” refers to the molar ratio of the first and second isocyanate-based compounds included in the second elastomer film layer 120, 220.

For example, the molar ratio refers to a ratio between a first isocyanate-based compound and a second isocyanate-based compound included in an elastomer film layer comprising a self-healing polymer (SHP) material. The molar ratio may be 0.1:0.9, 0.2:0.8, 0.3:0.7, 0.4:0.6, 0.5:0.5, 0.6:0.4, 0.7:0.3, 0.8:0.2, or 0.9:0.1, but is not limited thereto.

In this case, the molar ratio of the first isocyanate-based compound and the second isocyanate-based compound may be controlled such that the total sum is 1 during synthesis, with the molar ratio of each segment adjusted to maintain a total of 1. The mechanical properties and self-healing characteristics may be controlled according to the adjusted molar ratio.

The isocyanate-based compound comprises a first isocyanate-based compound and a second isocyanate-based compound. Specifically, the isocyanate-based compound included in the self-healing adhesive patch 100, 200 for an electronic skin sensor may include a first isocyanate-based compound that forms strong hydrogen bonding to enhance mechanical properties, and a second isocyanate-based compound that forms weak hydrogen bonding to improve self-healing characteristics. Preferably, the bonding energy of the hydrogen bonding may be in a range of 0 KJ/mol to 150 KJ/mol, and the strength of the hydrogen bonding (i.e., strong vs. weak) may be determined relatively.

For example, when the first isocyanate-based compound is 4,4′-methylenebis (phenyl isocyanate) (MPI) and the second isocyanate-based compound is isophorone diisocyanate (IPDI), the 4,4′-methylenebis (phenyl isocyanate) (MPI) may include four sites capable of forming hydrogen bonds, and the isophorone diisocyanate (IPDI) may include two sites capable of forming hydrogen bonds.

Specifically, 4,4′-methylenebis (phenyl isocyanate) (MPI) can form hydrogen bonds through all four N—H groups with adjacent MPI units without being significantly affected by steric hindrance. In contrast, isophorone diisocyanate (IPDI), due to steric effects, can form hydrogen bonds through only two of its four N—H groups with adjacent IPDI units.

Accordingly, MPI is capable of forming relatively strong hydrogen bonds compared to IPDI, while IPDI forms relatively weaker hydrogen bonds compared to MPI.

In addition, the first elastomer film layer and the second elastomer film layer may be configured to control their respective Young's moduli by adjusting hydrogen bonding between the polymer compound and the isocyanate-based compound. Preferably, the Young's modulus of the first elastomer film layer and the second elastomer film layer may be controlled in a range of 0.1 MPa to 10 MPa.

Hydrogen bonding refers to bonding between molecules and proceeds through a reversible process of bonding and dissociation. When a difference in Young's modulus arises due to hydrogen bonding, the elasticity within the polymer may temporarily vary. However, over time, the hydrogen bonds can be restored, thereby recovering elasticity and providing a self-healing effect.

In this context, Young's modulus refers to an elastic modulus that defines the relationship between stress (force per unit area) and strain in the uniaxial deformation region of a linear elastic material.

For example, when the first isocyanate-based compound is 4,4′-methylenebis (phenyl isocyanate) (MPI) and the second isocyanate-based compound is isophorone diisocyanate (IPDI), if the molar ratio of MPI is greater than that of IPDI within the elastomer film layer, the Young's modulus of the self-healing elastomer may increase. As the Young's modulus increases, the elastic modulus also increases, thereby enhancing the self-healing effect.

That is, the self-healing adhesive patch 100, 200 for an electronic skin sensor according to one embodiment of the present invention may be configured to control hydrogen bonding between the polymer compound and the isocyanate-based compound of the first elastomer film layer 110, 210 and the polymer compound and the isocyanate-based compound of the second elastomer film layer 120, 220. By adjusting the hydrogen bonding, both the mechanical properties and self-healing characteristics of the self-healing adhesive patch 100, 200 can be simultaneously ensured.

The first molar ratio of the first isocyanate-based compound to the second isocyanate-based compound included in the first elastomer film layer 110, 210 may be in a range of 0.1:0.9 to 0.9:0.1. Preferably, the first molar ratio may be 0.6:0.4. If the first molar ratio is less than 0.6:0.4, there may be a problem of degraded mechanical properties, and if the first molar ratio exceeds 0.6:0.4, there may be a problem of degraded self-healing characteristics.

The second molar ratio of the first isocyanate-based compound to the second isocyanate-based compound included in the second elastomer film layer 120, 220 may also be in a range of 0.1:0.9 to 0.9:0.1. Preferably, the second molar ratio may be 0.8:0.2. If the second molar ratio is less than 0.8:0.2, there may be a problem of degraded mechanical properties, and if the second molar ratio exceeds 0.8:0.2, there may be a problem of degraded self-healing characteristics.

The thickness ratio of the first elastomer film layer 110, 210 to the second elastomer film layer 120, 220 may be in a range of 1:2 to 2:1. If the thickness ratio is less than 1:2, there may be a problem of degraded mechanical properties, and if the thickness ratio exceeds 2:1, there may be a problem of degraded self-healing characteristics.

Preferably, the thickness ratio of the first elastomer film layer 110, 210 to the second elastomer film layer 120, 220 may be 1:1. A self-healing adhesive patch 100, 200 for an electronic skin sensor employing this thickness ratio may provide both self-healing characteristics and mechanical properties.

In addition, a self-healing adhesive patch 100, 200 for an electronic skin sensor according to one embodiment of the present invention may comprise: a first elastomer film layer 110, 210 comprising a polymer compound and an isocyanate-based compound; a second elastomer film layer 120, 220 formed on the first elastomer film layer 110, 210 and comprising a polymer compound and an isocyanate-based compound; and a third elastomer film layer 230 formed on the second elastomer film layer 120, 220 and comprising a polymer compound and an isocyanate-based compound. The isocyanate-based compound may comprise a first isocyanate-based compound and a second isocyanate-based compound.

In particular, in the self-healing adhesive patch 200 for an electronic skin sensor according to one embodiment of the present invention, a third molar ratio of the first isocyanate-based compound to the second isocyanate-based compound in the third elastomer film layer 230 may be different from the first molar ratio and the second molar ratio.

Here, the third molar ratio refers to a molar ratio of the first isocyanate-based compound and the second isocyanate-based compound included in the third elastomer film layer 230.

In addition, the third elastomer film layer may be configured to control its Young's modulus by adjusting hydrogen bonding between the polymer compound and the isocyanate-based compound. Preferably, the Young's modulus of the third elastomer film layer may be controlled in a range of 0.1 MPa to 10 MPa.

Furthermore, the third elastomer film layer 230 may be formed between the second elastomer film layer 120, 220 and the electrode 130, 240.

The third molar ratio of the first isocyanate-based compound to the second isocyanate-based compound included in the third elastomer film layer 230 may be in a range of 0.1:0.9 to 0.9:0.1. Preferably, the third molar ratio may be 0.2:0.8. If the third molar ratio is less than 0.2:0.8, there may be a problem of degraded mechanical properties, and if the third molar ratio exceeds 0.2:0.8, there may be a problem of degraded self-healing characteristics.

The thickness of the third elastomer film layer 230 may be in a range of 20 μm to 30 μm.

The third elastomer film layer 230 may be formed with a reduced thickness ratio compared to the thicknesses of the first elastomer film layer 110, 210 and the second elastomer film layer 120, 220, so as to maintain the elasticity and micro-patterning of the overall self-healing adhesive patch 100, 200 for an electronic skin sensor.

The polymer compound may comprise at least one selected from the group consisting of polydimethylsiloxane (PDMS), polyethylene oxide (PEO), perfluoropolyether (PFPE), polybutylene (PB), poly (ethylene-co-1-butylene), poly (butadiene), hydrogenated poly (butadiene), poly (ethylene oxide)-poly (propylene oxide) block copolymer, and poly (hydroxyalkanoate). Preferably, the polymer compound may be polydimethylsiloxane (PDMS) represented by Chemical Formula 1.

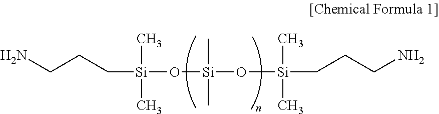

For example, polydimethylsiloxane (PDMS), represented by Chemical Formula 1, may provide elasticity to the self-healing adhesive patch 100, 200 and may facilitate fixation of the isocyanate-based compound within the self-healing adhesive patch 100, 200. In addition, polydimethylsiloxane (PDMS) exhibits hydrophobic properties due to its low surface energy, making it suitable for use in underwater environments.

The isocyanate-based compound may comprise at least one selected from the group consisting of 4,4′-methylenebis (phenyl isocyanate) (MPI), isophorone diisocyanate (IPDI), cyclohexylene diisocyanate, hydrogenated toluene diisocyanate (hydrogenated TDI), bis(2-isocyanatoethyl)-4-diclohexene-1,2-dicarboxylate, 2,5-norbornene diisocyanate, and 2,6-norbornene diisocyanate.

The first isocyanate-based compound refers to an isocyanate-based compound that forms strong hydrogen bonds, and the second isocyanate-based compound may refer to an isocyanate-based compound that forms weak hydrogen bonds.

The bonding energy of the hydrogen bonds may be in a range of 0 KJ/mol to 150 KJ/mol. The classification between strong and weak hydrogen bonds may be determined relatively, and may depend on the steric structure of the isocyanate-based compounds.

For example, the first isocyanate-based compound may be 4,4′-methylenebis (phenyl isocyanate) (MPI), represented by Chemical Formula 2, which forms strong hydrogen bonds. The second isocyanate-based compound may be isophorone diisocyanate (IPDI), represented by Chemical Formula 3, which forms weak hydrogen bonds.

MPI can form hydrogen bonds through four N—H groups with adjacent MPI units without significant steric hindrance, whereas IPDI, due to steric effects, can form hydrogen bonds with adjacent IPDI units through only two of its four N—H groups. Accordingly, MPI is capable of forming relatively stronger hydrogen bonds compared to IPDI.

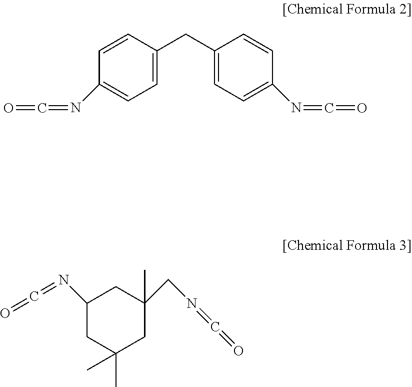 [SMILES: O=C=Nc1ccc(Cc2ccc(N=C=O)cc2)cc1 ; CC1(C)CC(N=C=O)CC(C)(CN=C=O)C1]

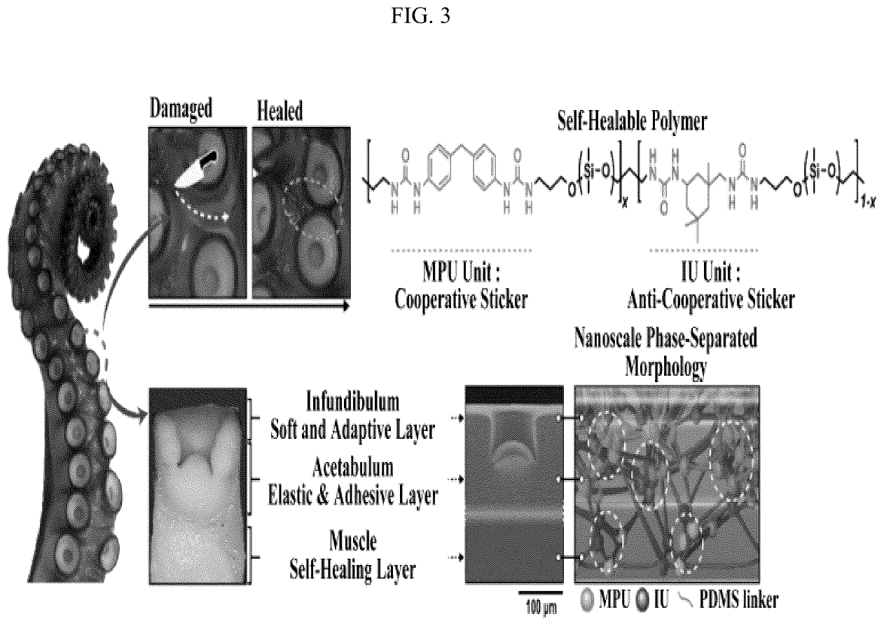

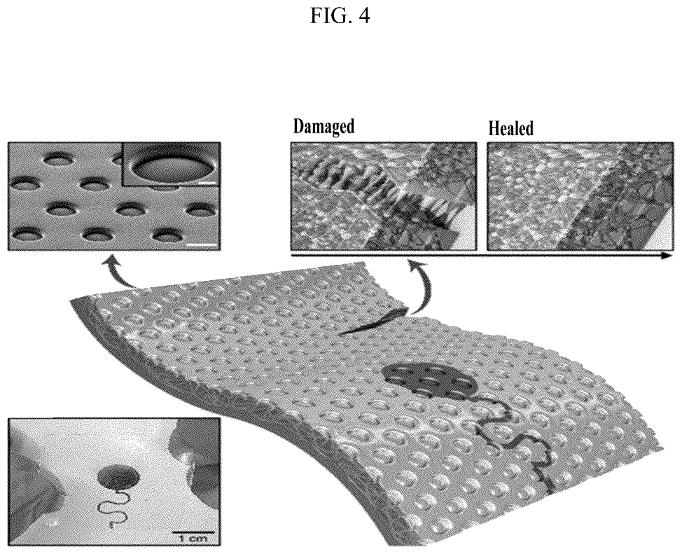

FIGS. 3 and 4 are schematic diagrams illustrating a self-healing mechanism of a self-healing adhesive patch for an electronic skin sensor according to one embodiment of the present invention.

Referring to FIGS. 3 and 4, the self-healing adhesive patch for an electronic skin sensor according to one embodiment of the present invention may comprise a self-healing polymer (SHP) including polydimethylsiloxane (PDMS) and both 4,4′-methylenebis (phenyl isocyanate) (MPI) and isophorone diisocyanate (IPDI).

The self-healing function may occur through dynamic bonding, which, unlike well-known covalent bonding, may involve secondary bonding such as hydrogen bonding. Secondary bonding is generally formed at lower bonding energy than covalent bonds, and thus can be more easily broken and reformed. Therefore, when fractured surfaces are brought back into contact by external force, the dynamic bonding can be re-established, allowing the material to recover its original state and exhibit self-healing properties.

In addition, the self-healing adhesive patch for an electronic skin sensor according to one embodiment of the present invention may implement a trilayer structure inspired by the muscle layer, acetabulum layer, and infundibulum layer of the octopus (Octopus vulgaris) suction cup. Specifically, the patch may apply the self-healing characteristics of the muscle layer, the elasticity and adhesiveness of the acetabulum layer, and the flexibility and adaptability of the infundibulum layer.

For example, the self-healing adhesive patch for an electronic skin sensor according to one embodiment of the present invention may implement both the morphology and function of an octopus suction cup protuberance by incorporating PDMS with MPI and IPDI, wherein such implementation may be achieved by adjusting the molar ratio of MPI to IPDI.

As the ratio of MPI, which forms relatively strong hydrogen bonds, increases, the mechanical properties of the self-healing adhesive patch may be enhanced. Conversely, as the ratio of IPDI, which forms relatively weak hydrogen bonds, increases, the self-healing efficiency of the patch may be improved.

Accordingly, the self-healing adhesive patch for an electronic skin sensor according to one embodiment of the present invention can realize the structure and function of the suction cup protuberance of an octopus by adjusting the molar ratio of MPI and IPDI in PDMS, thereby providing improved mechanical properties and enhanced self-healing efficiency.

The self-healing adhesive patch for an electronic skin sensor according to one embodiment of the present invention may comprise a polymer compound represented by Chemical Formula 1 and isocyanate-based compounds represented by Chemical Formulae 2 and 3. Using Chemical Formulae 1, 2, and 3, an elastomer film layer comprising a self-healing polymer (SHP) material represented by Chemical Formula 4 may be synthesized.

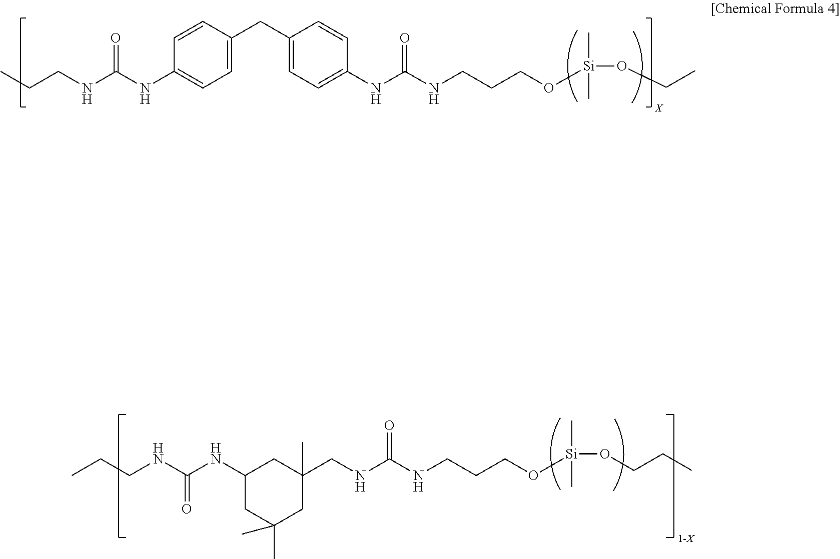

The elastomer film layer comprising a self-healing polymer (SHP) material represented by Chemical Formula 4 may include 4,4′-methylenebis (phenyl isocyanate) (MPI), which is the first isocyanate-based compound represented by Chemical Formula 2, and isophorone diisocyanate (IPDI), which is the second isocyanate-based compound represented by Chemical Formula 3. The molar ratio of X:1−X in Chemical Formula 4 may be 0.1:0.9, 0.2:0.8, 0.3:0.7, 0.4:0.6, 0.5:0.5, 0.6:0.4, 0.7:0.3, 0.8:0.2, or 0.9:0.1.

The molar ratio of the first isocyanate-based compound (MPI) and the second isocyanate-based compound (IPDI) may be controlled such that the total sum is 1 during synthesis, and the total molar ratio of each segment may also be adjusted to 1. The mechanical properties and self-healing characteristics may be controlled according to the adjusted molar ratio.

For example, when the molar ratio of MPI to IPDI is 0.6:0.4, the elastomer film layer may exhibit improved mechanical properties compared to the case in which the molar ratio is 0.2:0.8.

Accordingly, the self-healing adhesive patch for an electronic skin sensor according to one embodiment of the present invention may include the first isocyanate-based compound MPI represented by Chemical Formula 2 and the second isocyanate-based compound IPDI represented by Chemical Formula 3, and may control the mechanical properties and self-healing characteristics by adjusting the molar ratio between the first and second isocyanate-based compounds.

For example, the self-healing adhesive patch may include a first elastomer film layer containing MPI and IPDI at a molar ratio of 0.6:0.4, a second elastomer film layer containing MPI and IPDI at a molar ratio of 0.8:0.2, and a third elastomer film layer containing MPI and IPDI at a molar ratio of 0.2:0.8.

The first elastomer film layer containing MPI and IPDI at a molar ratio of 0.6:0.4 may exhibit high self-healing efficiency. The second elastomer film layer containing MPI and IPDI at a molar ratio of 0.8:0.2 may facilitate pattern formation. The third elastomer film layer containing MPI and IPDI at a molar ratio of 0.2:0.8 may provide conformal contact with human skin.

In addition, the elastomer film layer may include the first isocyanate-based compound and the second isocyanate-based compound dispersed in a nanoscale phase within the polymer compound.

The nanoscale phase-separated structure may be achieved when the first isocyanate-based compound and the second isocyanate-based compound are incorporated into the backbone chain of the polymer compound. In such a structure, the first isocyanate-based compound may cooperatively interact with adjacent units of the same compound and form non-covalent bonds, thereby inducing nanoscale phase separation and improving the mechanical properties.

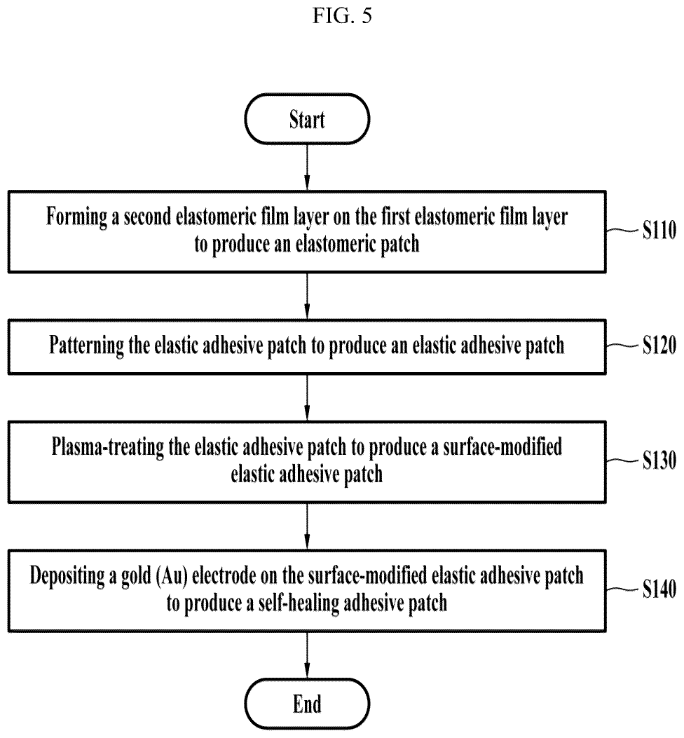

FIG. 5 is a flowchart illustrating a method for manufacturing a self-healing adhesive patch for an electronic skin sensor according to one embodiment of the present invention.

The method for manufacturing a self-healing adhesive patch for an electronic skin sensor according to an embodiment of the present invention may include the same components as the self-healing adhesive patch for an electronic skin sensor described in the embodiment above, and a redundant description of identical components will be omitted.

The method for manufacturing a self-healing adhesive patch for an electronic skin sensor according to one embodiment of the present invention includes: a step (S110) of forming a second elastomer film layer on a first elastomer film layer to fabricate an elastomer patch, a step (S120) of patterning the elastomer patch to fabricate an elastic adhesive patch, a step (S130) of performing plasma surface treatment on the elastic adhesive patch to fabricate a surface-modified elastic adhesive patch, and a step (S140) of depositing an electrode on the surface-modified elastic adhesive patch to fabricate the self-healing adhesive patch.

First, the method for manufacturing a self-healing adhesive patch for an electronic skin sensor according to one embodiment of the present invention proceeds with a step (S110) of forming a second elastomer film layer on a first elastomer film layer to fabricate an elastomer patch.

The step (S110) of fabricating the elastomer patch may include: a step (S111) of forming the first elastomer film layer on a substrate, a step (S112) of forming the second elastomer film layer on the first elastomer film layer, and a step (S113) of performing heat treatment on the first and second elastomer film layers.

The step (S111) of forming the first elastomer film layer may include: preparing a polymer compound solution containing a polymer compound, adding a first isocyanate-based compound and a second isocyanate-based compound to the polymer compound solution to prepare a first elastomer solution, and coating the first elastomer solution on a substrate to form the first elastomer film layer.

Subsequently, the step (S112) of forming the second elastomer film layer may include: preparing a polymer compound solution containing a polymer compound, adding the first isocyanate-based compound and the second isocyanate-based compound to the polymer compound solution to prepare a second elastomer solution, and coating the second elastomer solution on the substrate to form the second elastomer film layer.

The first molar ratio of the first isocyanate-based compound and the second isocyanate-based compound included in the first elastomer film layer may be in a range of 0.1:0.9 to 0.9:0.1. Preferably, the first molar ratio may be 0.6:0.4. If the ratio is less than 0.6:0.4, there may be a problem of degraded mechanical properties, and if the ratio exceeds 0.6:0.4, there may be a problem of degraded self-healing characteristics.

The second molar ratio of the first isocyanate-based compound and the second isocyanate-based compound included in the second elastomer film layer may also be in a range of 0.1:0.9 to 0.9:0.1. Preferably, the second molar ratio may be 0.8:0.2. If the ratio is less than 0.8:0.2, there may be a problem of degraded mechanical properties, and if the ratio exceeds 0.8:0.2, there may be a problem of degraded self-healing characteristics.

The step (S113) of performing heat treatment may include: a step of applying pressure to the first and second elastomer film layers, and a step of thermally treating the compressed first and second elastomer film layers.

In this case, the step of applying pressure to the first and second elastomer film layers may form conformal contact between the layers. The subsequent heat treatment may relieve stress in the stacked layers.

Preferably, the heat treatment may be performed for 1 to 5 minutes. If the treatment time is less than 1 minute, stress relaxation may be insufficient; and if it exceeds 5 minutes, the layered structure of the patch may collapse.

In addition, the step (S110) of fabricating the elastomer patch may further include: a step (S111) of forming a first elastomer film layer including the first molar ratio, a step (S112) of forming a second elastomer film layer including the second molar ratio on the first elastomer film layer, and a step (S113) of forming a third elastomer film layer including the third molar ratio.

In the step (S110) of fabricating the elastomer patch, the third molar ratio of the first isocyanate-based compound and the second isocyanate-based compound included in the third elastomer film layer may be in a range of 0.1:0.9 to 0.9:0.1. Preferably, the third molar ratio may be 0.2:0.8. If the third molar ratio is less than 0.2:0.8, there may be a problem of degraded mechanical properties, and if it exceeds 0.2:0.8, there may be a problem of degraded self-healing characteristics.

Subsequently, the method for manufacturing a self-healing adhesive patch for an electronic skin sensor according to one embodiment of the present invention proceeds with a step (S120) of patterning the elastomer patch to fabricate an elastic adhesive patch.

In the step (S120) of fabricating the elastic adhesive patch, the patterning may be performed using one method selected from the group consisting of imprinting, 3D printing, and contact printing. Preferably, the patterning method may be imprinting.

Nanoimprinting is a micro- and nanofabrication process in which a pattern defined on a template is transferred to a target substrate, typically generating nanoscale patterns on a very thin polymer film. It is one of the most common techniques used to process polymer structures.

The imprinting process can minimize damage to the material and the structure by utilizing low pressure and temperature, and allows the formation of the desired shape through a pre-fabricated mold with ease.

Specifically, pillar patterns formed through imprinting can induce Laplace pressure and interfacial tension via interchain interactions, leading to pattern deformation. When a mold having fine patterns is physically brought into contact with a polymer layer and pressure is applied, creep deformation causes the polymer to conform to the shape of the mold over time, thereby forming the desired pattern.

To function effectively as a skin-attachable device, the self-healing adhesive patch for an electronic skin sensor must maintain stable conformal contact and interfacial adhesion on curved and rough skin surfaces. The self-healing adhesive patch for an electronic skin sensor according to one embodiment of the present invention may provide excellent conformal contact and leakage-free suction performance by patterning the third elastomer film layer using an imprinting process.

In particular, the self-healing adhesive patch for an electronic skin sensor according to one embodiment of the present invention may deliver significant suction performance under both dry and underwater conditions by implementing an octopus-inspired pattern through patterning.

Subsequently, the method for manufacturing a self-healing adhesive patch for an electronic skin sensor according to one embodiment of the present invention proceeds with a step (S130) of performing plasma surface treatment on the elastic adhesive patch to fabricate a surface-modified elastic adhesive patch.

In the step (S130) of fabricating the surface-modified elastic adhesive patch, preferably, oxygen (O₂) plasma treatment is employed. The oxygen (O₂) plasma treatment may be performed at a power level of 10 W to 100 W for a duration of 10 seconds to 30 minutes. If the power is less than 10 W, the plasma effect may be negligible. If the power exceeds 100 W, the material may be adversely affected. If the treatment duration is less than 10 seconds, electrode formation may be insufficient, and if it exceeds 30 minutes, the formed electrode may undergo oxidation or severe cracking.

The step (S130) of fabricating the surface-modified elastic adhesive patch may increase the surface energy of the elastic adhesive patch by oxygen (O₂) plasma treatment, thereby enabling efficient deposition of electrodes at the interface.

The self-healing adhesive patch for an electronic skin sensor according to one embodiment of the present invention may improve the adhesion at the metal-polymer interface by saturating the surface energy through oxygen (O₂) plasma treatment, thus providing favorable conditions for electrode deposition.

Finally, the method for manufacturing a self-healing adhesive patch for an electronic skin sensor proceeds with a step (S140) of depositing an electrode onto the surface-modified elastic adhesive patch to fabricate the self-healing adhesive patch.

The electrode may comprise at least one selected from the group consisting of gold (Au), copper (Cu), aluminum (Al), iron (Fe), magnesium (Mg), titanium (Ti), silver (Ag), ruthenium (Ru), platinum (Pt), iridium (Ir), molybdenum (Mo), palladium (Pd), zinc (Zn), silicon (Si), nickel (Ni), tin (Sn), tungsten (W), calcium (Ca), potassium (K), sodium (Na), amorphous indium-gallium-zinc oxide (a-IGZO), and germanium (Ge). Preferably, the electrode may be gold (Au).

The gold (Au) electrode may be deposited to a thickness of 30 nm to 100 nm. If the thickness is less than 30 nm, electrical conductivity may be significantly reduced, and if the thickness exceeds 100 nm, non-uniform electrode films may form.

The electrode may be deposited on the elastic adhesive patch by physical vapor deposition (PVD), sputtering, electron beam deposition, or by oxidation-reduction methods.

In the step (S140) of fabricating the self-healing adhesive patch, a gold (Au) electrode was physically vapor-deposited onto the third elastomer film layer of the surface-modified elastic adhesive patch. As a result, the patch may function as a sensor capable of detecting electrocardiogram (ECG) signals even under dry or underwater conditions and during body movements, including when the patch is partially damaged.

Accordingly, the self-healing adhesive patch for an electronic skin sensor according to one embodiment of the present invention, and the method for manufacturing the same, may provide improved mechanical properties, self-healing characteristics, and adhesion properties by stacking multiple elastomer film layers—each comprising a polymer compound and an isocyanate-based compound—having controlled molar ratios.

In addition, by patterning the adhesive surface and depositing an electrode thereon, the self-healing adhesive patch for an electronic skin sensor may maintain excellent pattern fidelity and self-healing performance. Furthermore, the patch may be applied as a biosensor that stably measures biosignals even under various environmental conditions, body movements, and physical damage, while retaining its original properties. A manufacturing method and the patch fabricated thereby are provided.

Hereinafter, the present invention will be described in further detail through exemplary embodiments. These embodiments are provided for illustrative purposes only and are not intended to limit the scope of the present invention.

### Preparation Example 1-1: Synthesis of Self-Healing Polymer Elastomer (SHP)

Amino-terminated polydimethylsiloxane (H₂N-PDMS-NH₂, MW=5000, 50 g) was dissolved in chloroform (CF, 300 mL) and stirred for 1 hour under a nitrogen (N₂) atmosphere, followed by lowering the temperature to 0° C. Triethylamine (Et₃N, 5 mL) was added to the solution and stirred for an additional hour. After stirring, 4,4′-methylenebis (phenyl isocyanate) (MPI) (0.5-2 g, 0.2 eq-0.8 eq) and isophorone diisocyanate (IPDI) (1.8-0.45 g, 0.8 eq-0.2 eq) were added to the CF and mixed. The resulting solution was stirred at 0° C. for 1 hour at an appropriate speed to ensure uniform mixing throughout.

The temperature was then gradually raised to room temperature, and the mixture was stirred continuously for 3 days.

The resulting self-healing polymer elastomer (SHP) was purified through an evaporation-precipitation-dissolution process. Before purification, methanol (10 mL) was added to the reaction solution and stirred for 30 minutes to remove unreacted isocyanate. The solvent was then evaporated using a rotary evaporator under reduced pressure until half of the total volume remained. The concentrated solution was left to stand until the precipitate settled at the bottom, after which the supernatant was removed and chloroform (CF, 50 mL) was added to dissolve the remaining residue. This process was repeated three times.

Finally, the resulting mixture was poured into a Teflon mold and vacuum-dried overnight to remove residual solvents, thereby synthesizing the self-healing polymer elastomer (SHP).

### Preparation Example 1-2: Fabrication of Self-Healing Elastomer Film Layer (PDMS-MPUₓ-IU₁, Film)

3 to 5 g of the self-healing polymer elastomer (SHP) synthesized in Preparation Example 1-1 was dissolved in 15-20 mL of chloroform (CHCl₃) and stirred at 50° C. The resulting viscous solution was stirred for more than 3 hours and then slowly cooled to room temperature. The prepared solution was cast onto an OTS-treated 4-inch silicon (Si) substrate and dried at room temperature for 6 hours, followed by vacuum drying (approximately 100 Torr) at 80° C. for 3 hours. The resulting polymer film was then cut into specific dimensions and detached from the substrate for mechanical testing.

### Example 1-1: Fabrication of a Triple-Layer Self-Healing Adhesive Patch (SOIA)

PDMS-MPU_{0.6}-IU_{0.4} and PDMS-MPU_{0.8}-IU_{0.2} films prepared according to Preparation Example 1-2 were laminated at thickness ratios of 2:1, 1:1, and 1:2. Conformal contact between the two films was established using a pressing machine for 1 hour to eliminate trapped air bubbles. The compressed bilayer films were placed on a hot plate and heated for 1 minute to induce stress relaxation, thereby fabricating a bilayer self-healing adhesive patch (SOIA). Subsequently, PDMS-MPU_{0.4}-IU_{0.6} film was spin-coated onto an OTS-treated silicon (Si) substrate and transferred onto the top of the PDMS-MPU_{0.8}-IU_{0.2} film to fabricate a triple-layer self-healing adhesive patch (d-SOIA).

### Example 1-2: Fabrication of a Triple-Layer Self-Healing Adhesive Patch (d-SOIA)

PDMS-MPU_{0.6}-IU_{0.4} and PDMS-MPU_{0.8}-IU_{0.2} films prepared according to Preparation Example 1-2 were laminated at thickness ratios of 2:1, 1:1, and 1:2. Conformal contact between the two films was achieved using a pressing machine for 1 hour to eliminate air bubbles. After compression, the bilayer film was placed on a hot plate and heated for 1 minute to induce stress relaxation, thereby fabricating a bilayer self-healing adhesive patch (SOIA). Subsequently, PDMS-MPU_{0.6}-IU_{0.4} film was spin-coated onto an OTS-treated silicon (Si) substrate and transferred onto the top of the PDMS-MPU_{0.6}-IU_{0.4} film to fabricate a triple-layer self-healing adhesive patch (d-SOIA).

### Example 2: Patterned Self-Healing Adhesive Patch (t-SOIA)

To fabricate an octopus-inspired pattern master, a silicon mold with microhole patterns (diameter: 100 μm; aspect ratio: 1) was prepared using photolithography followed by reactive ion etching. The mold was treated in an argon atmosphere using a 0.03 M solution of a fluorinated self-assembled monolayer (FOTCS, Samchun Chemical Co.) diluted in anhydrous heptane.

An elastic PDMS precursor was poured into the prepared mold and thermally cured at 80° C. for 2 hours to obtain a PDMS patch featuring the octopus-inspired structure. To integrate this patch with Example 1-1 and Example 1-2, the polymer film on a substrate was pressed using an OIA reverse mold made of PDMS at 80° C. and left for 2 hours. Thereafter, the PDMS mold was removed to yield a patterned self-healing adhesive patch (t-SOIA) with a film thickness of less than 400 μm.

### Example 3: Self-Healing Adhesive Patch with Gold (Au) Electrode (t-SOIA)

The t-SOIA patch obtained from Example 2 was subjected to oxygen (O₂) plasma surface treatment at a power of 10 W to 100 W for 10 seconds to 30 minutes to achieve surface modification. Following surface treatment, a gold (Au) electrode was deposited by physical vapor deposition onto the third layer (PDMS-MPU_{0.2}-IU_{0.8}) of the adhesive patch to a thickness of 30 nm to 100 nm.

Gold (Au, 99.9999%) was deposited onto the self-healing patch by thermal evaporation under a pressure below 1.0×10⁻⁶ Torr at a deposition rate of 2 Å/s. The deposition rate and thickness were monitored in situ using a quartz crystal microbalance. The deposited Au layer had a thickness ranging from 30 nm to 100 nm. During deposition, the substrate stage was rotated to ensure uniform film formation. After deposition, the sample was left to stabilize for approximately 30 minutes.

### Comparative Example 1: Flat Patch

A non-patterned flat patch was fabricated in the same manner as in Example 2, except that no patterning process was performed.

### Comparative Example 2: Commercial Electrocardiogram Sensor

A commercial electrocardiogram (ECG) sensor with a diameter of 2 cm, manufactured by Cardinal Health™, was used.

### Experimental Example 1: Evaluation of Mechanical Properties of the Self-Healing Polymer Elastomer

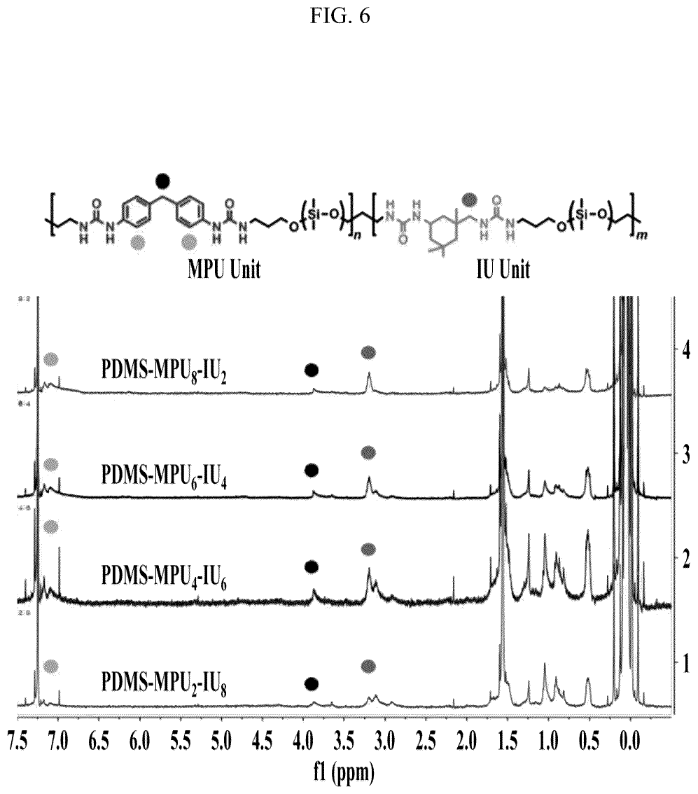

FIG. 6 illustrates the results of 1H nuclear magnetic resonance (NMR) spectroscopy analysis based on the molar ratio of MPU and IU.

Referring to FIG. 6, PDMS-MPU_{0.4}-IU_{0.6}, PDMS-MPU_{0.8}-IU_{0.2}, PDMS-MPU_{0.2}-IU_{0.8}, and PDMS-MPU_{0.6}-IU_{0.4}-synthesized by controlling the molar ratio of MPU and IU within the PDMS-based polymer—exhibited trend-wise changes in characteristic peaks of MPU and IU, depending on the synthesis ratio in the NMR analysis. These results confirm that the polymer elastomers were successfully synthesized.

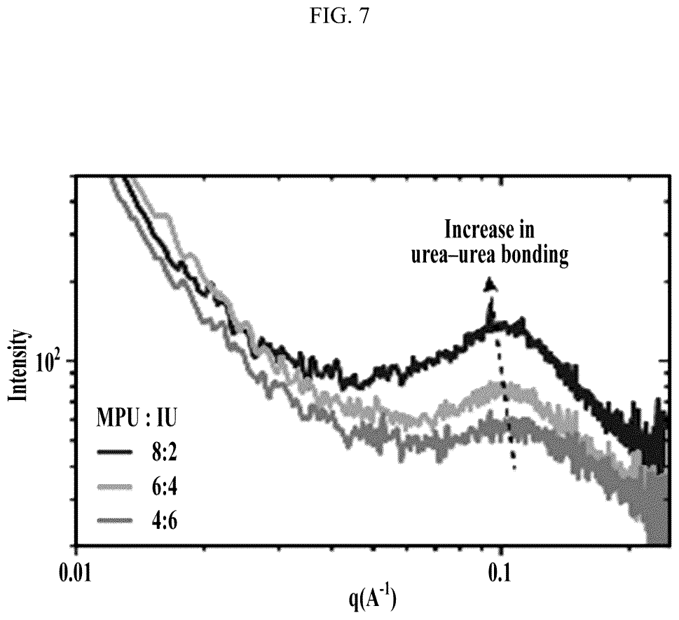

FIG. 7 shows the results of small-angle X-ray scattering (SAXS) analysis based on the molar ratio of MPU and IU.

Referring to FIG. 7, SAXS was performed to analyze the microstructure of the polymers, which is a method for determining the average particle size, shape, and domain structure of a material. The SAXS analysis showed that all polymer samples exhibited a distinct peak corresponding to a domain size in the range of 6 to 7 nm.

Additionally, the analysis confirmed an increase in urea-urea bonding in PDMS-MPU_{0.4}-IU_{0.6}, PDMS-MPU_{0.8}-IU_{0.2}, and PDMS-MPU_{0.6}-IU_{0.4}, indicating that an increased MPU ratio results in a larger domain size due to stronger hydrogen bonding.

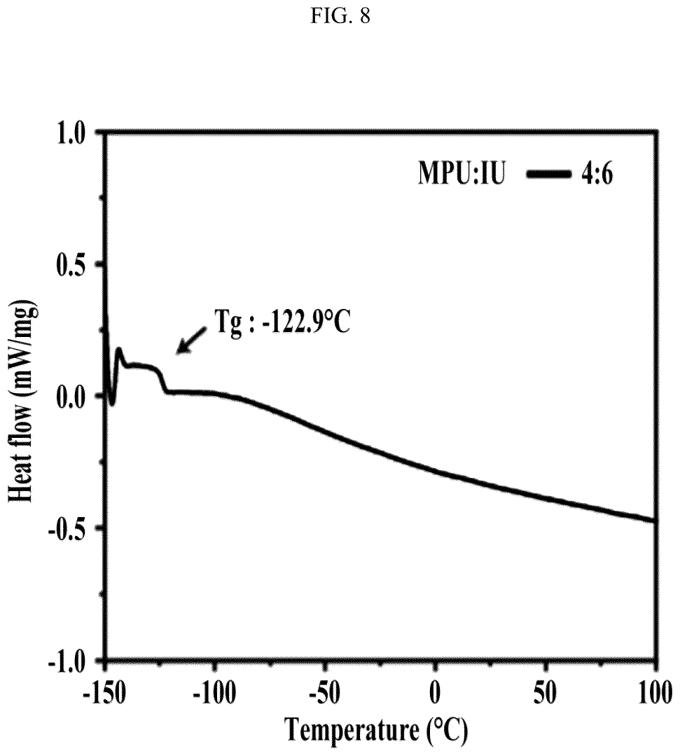

FIGS. 8 to 10 depict differential scanning calorimetry (DSC) results for the polymers with varying MPU and IU molar ratios.

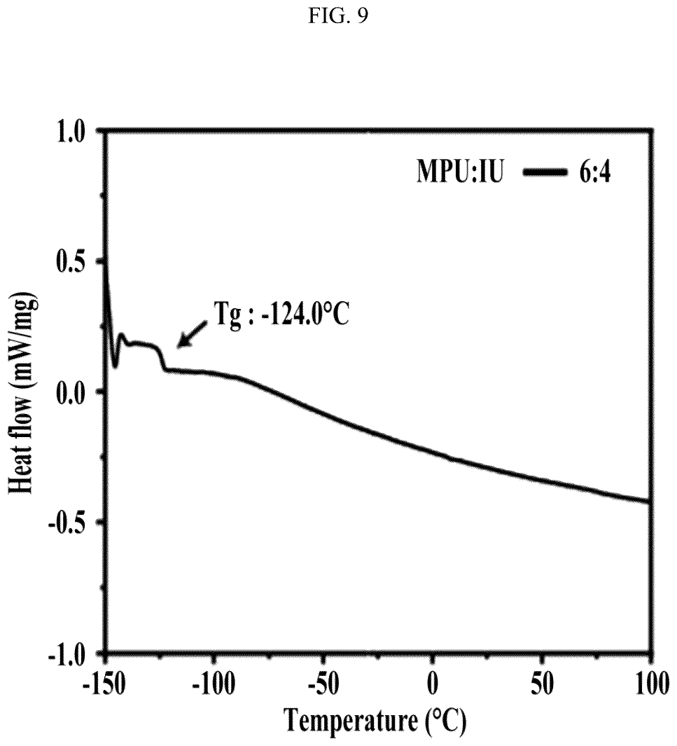

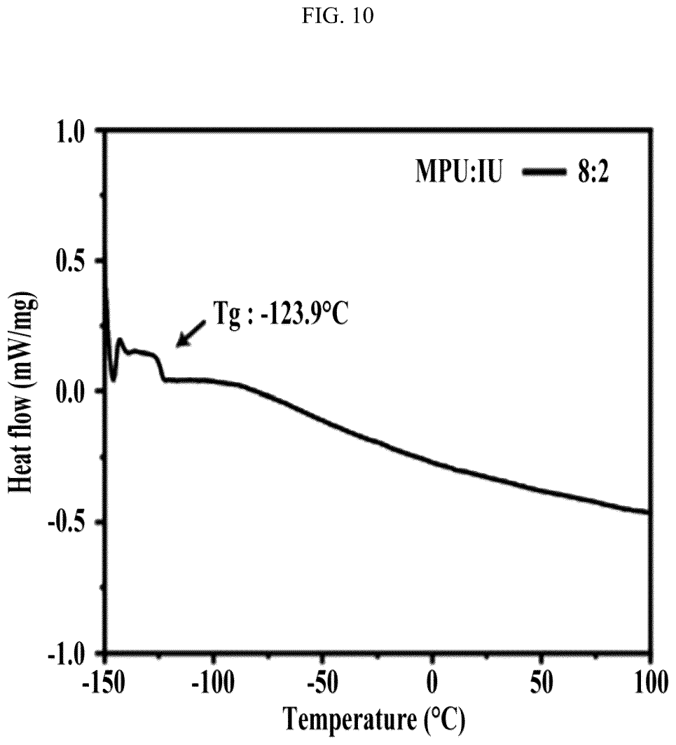

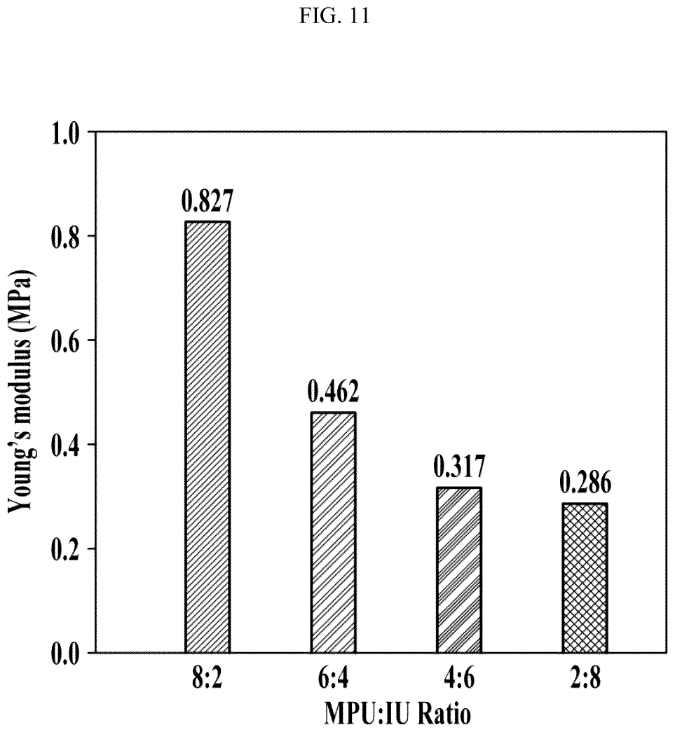

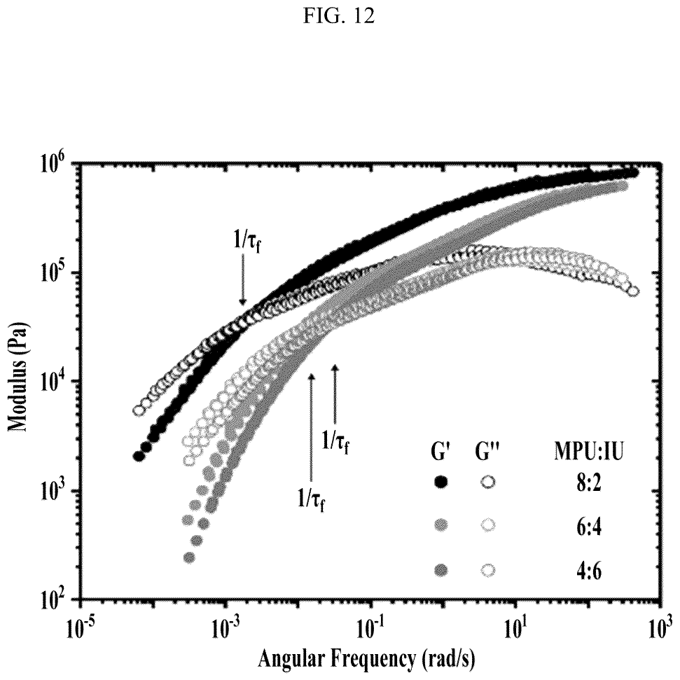

FIG. 8 shows the DSC result for PDMS-MPU_{0.4}-IU_{0.6}, FIG. 9 for PDMS-MPU_{0.6}-IU_{0.4}, and FIG. 10 for PDMS-MPU_{0.8}-IU_{0.2.}

Referring to FIGS. 8 to 10, DSC analysis was conducted by simultaneously heating and cooling the sample and reference materials to measure thermal transitions. The glass transition temperature (Tg) of each synthesized polymer was determined, and all compositions-including PDMS-MPU_{0.4}-IU_{0.6} (FIG. 8), PDMS-MPU_{0.6}-IU_{0.4} (FIG. 9), and PDMS-MPU_{0.8}-IU_{0.2} (FIG. 10)—exhibited Tg values below −120° C. These results confirm the potential for self-healing behavior at room temperature.

FIG. 11 illustrates a graph showing the Young's modulus according to the molar ratio of MPU and IU.

Referring to FIG. 11, the mechanical and rheological properties of the polymer elastomers synthesized with different MPU and IU molar ratios were measured using a universal testing machine (UTM). The Young's modulus values were determined to be 0.827 MPa for PDMS-MPU_{0.8}-IU_{0.2}, 0.462 MPa for PDMS-MPU_{0.6}-IU_{0.4}, 0.317 MPa for PDMS-MPU_{0.4}-IU_{0.6}, and 0.286 MPa for PDMS-MPU_{0.2}-IU_{0.8}, respectively. These results confirm that the Young's modulus of the self-healing elastomer increases with increasing MPU content.

FIG. 12 illustrates a graph evaluating the rheological behavior according to the molar ratio of MPU and IU.

Referring to FIG. 12, viscoelastic properties were evaluated using oscillatory rheology for PDMS-MPU_{0.4}-IU_{0.6}, PDMS-MPU_{0.8}-IU_{0.2}, and PDMS-MPU_{0.6}-IU_{0.4}. The crossover point between the loss modulus (G″) and storage modulus (G′) indicates the transition from liquid-like to solid-like behavior. The data indicate an increase in elastic moduli for all samples, demonstrating enhanced solid-like characteristics with increased MPU content.

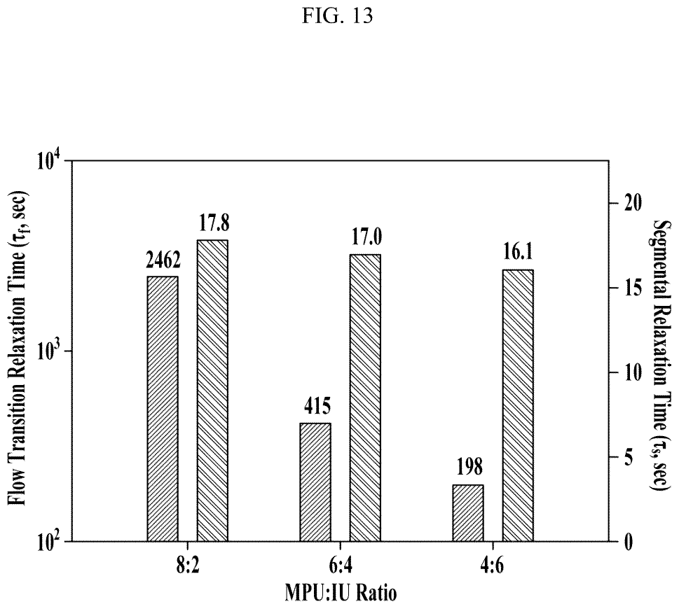

FIG. 13 shows a graph of flow transition relaxation time (τ_{f}) and segmental relaxation time (Ts) as a function of the molar ratio of MPU and IU.

Referring to FIG. 13, If was determined by observing the inverse frequency at the crossover point of G′/G″ between the terminal region and rubbery plateau. The τ_{f} values were measured as 2,462 seconds for PDMS-MPU_{0.8}-IU_{0.2, 415} seconds for PDMS-MPU_{0.6}-IU_{0.4}, and 198 seconds for PDMS-MPU_{0.4}-IU_{0.6}. This confirms that PDMS-MPU_{0.8}-IU_{0.2} behaves more like a glassy solid compared to PDMS-MPU_{0.6}-IU_{0.4}.

Furthermore, as the MPU content increases, the segmental relaxation time τₛ decreases. This is attributed to the increased urea-urea bonding and π-π stacking between MPU units, which interfere with chain rearrangement. These findings suggest that, at short timescales, PDMS-MPU_{0.4}-IU_{0.6} exhibits greater relaxation and flow behavior than PDMS-MPU_{0.8}-IU_{0.2}.

The segmental relaxation time (τₛ) was derived from the equations J′=G′/(η*ω)²=λ/[η*], where J′, G′, and [η*] represent the storage compliance, storage modulus, and complex viscosity, respectively.

Using the λ value at 0.05 rad·s⁻¹, it was observed that Ts remained similar across all MPU and IU molar ratios. This indicates that the segmental relaxation of MPU and IU is not correlated with the degradation of mechanical properties of the system.

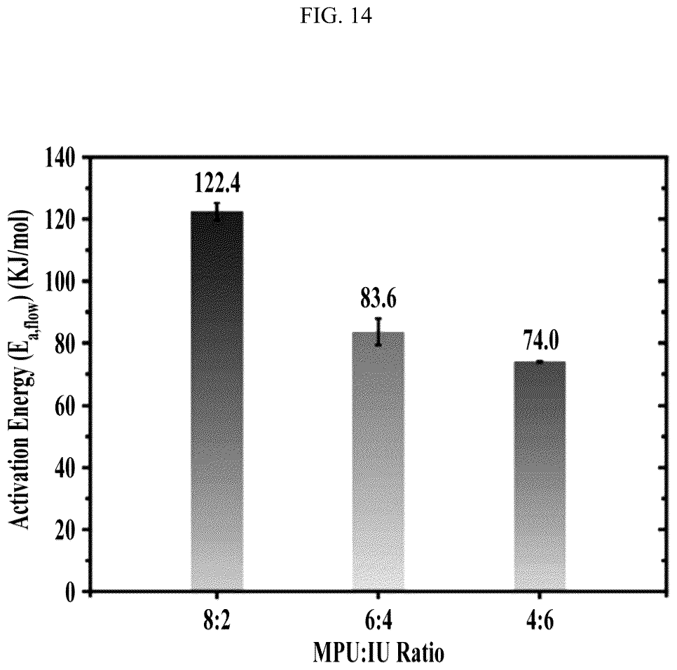

FIG. 14 is a graph comparing the activation energy (Eₐ,flow) according to the molar ratio of MPU and IU.

Referring to FIG. 14, the activation energy (Eₐ,flow) was estimated based on the Arrhenius-type temperature dependence demonstrated through mobility coefficients at various temperatures. As the MPU content increased, stronger hydrogen bonding between MPU units led to a higher activation energy (Eₐ,flow), resulting in increased resistance to flow.

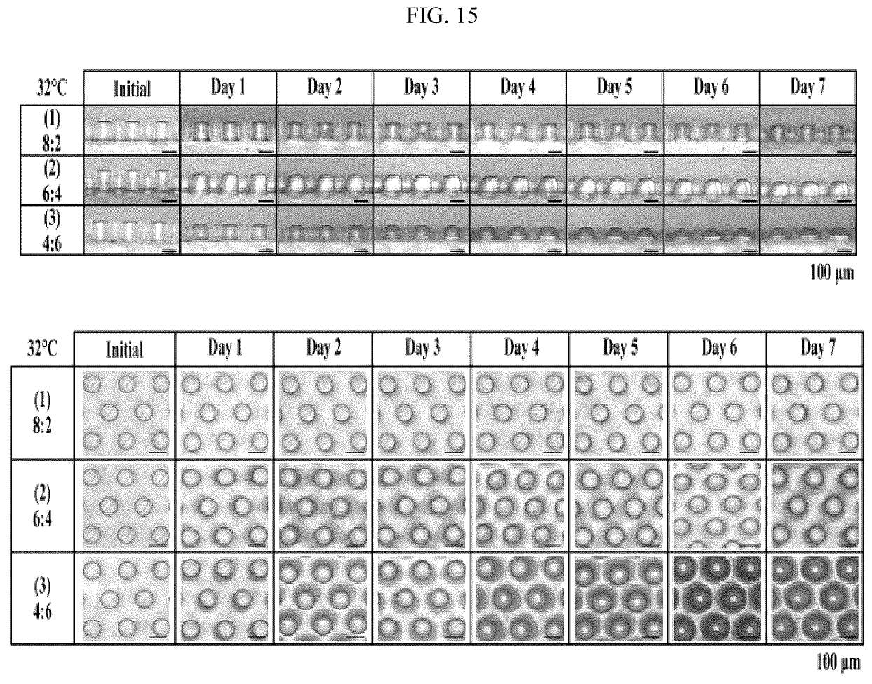

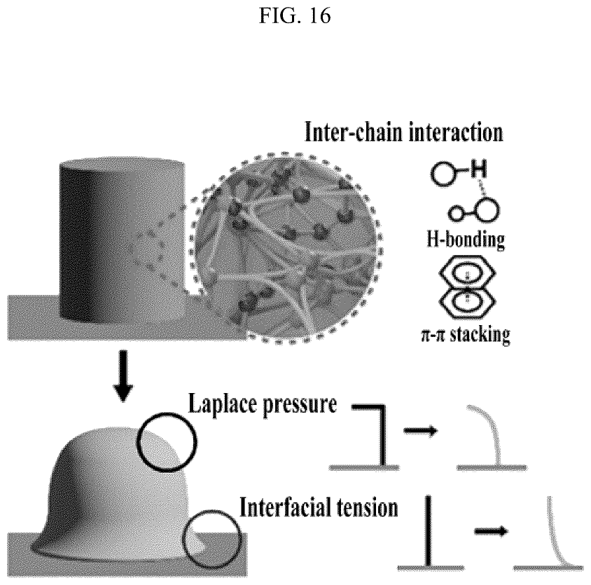

FIG. 15 shows side-view and top-view images illustrating the pattern retention behavior over time for different MPU and IU molar ratios, and FIG. 16 shows images of pattern reflow caused by the Laplace effect and interchain interactions.

Referring to FIGS. 15 and 16, the pattern structures formed on the substrate for PDMS-MPU_{0.4}-IU_{0.6}, PDMS-MPU_{0.6}-IU_{0.4}, and PDMS-MPU_{0.8}-IU_{0.2} had the same diameter of 100 μm and an aspect ratio (AR) of 1.3. However, each composition exhibited different reflow characteristics depending on the MPU to IU molar ratio. Experimental observations of the pattern morphology changes at 32° C. over a period of 7 days confirmed that higher MPU content results in slower pattern reflow and better retention of the original pattern structure.

Additionally, it was confirmed that the initial pillar patterns exhibited morphology changes induced by Laplace pressure and interfacial tension, which were generated through interchain interactions.

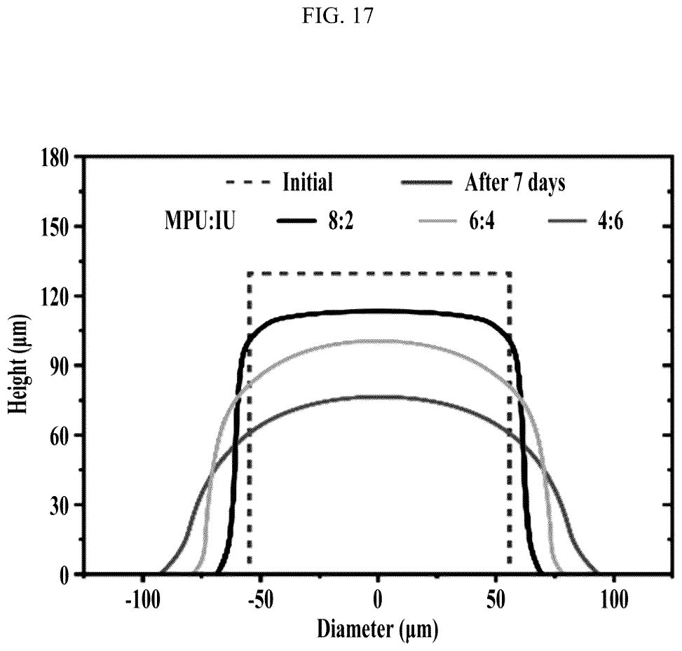

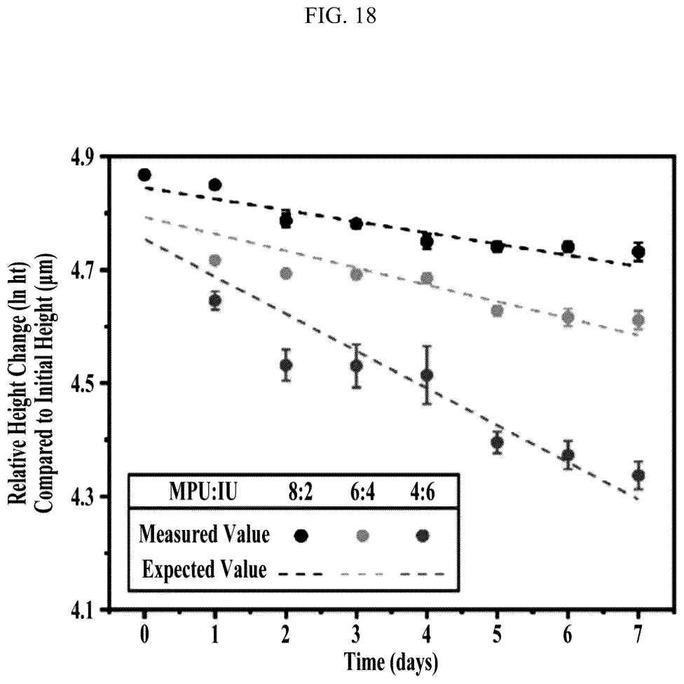

FIGS. 17 and 18 are graphs evaluating the structural stability over time according to the molar ratio of MPU and IU.

Referring to FIGS. 17 and 18, the structural stability was quantitatively evaluated by measuring the relative change in pattern height (In ht, μm) compared to the initial height. It was confirmed that as the MPU content increased in PDMS-MPU_{0.4}-IU_{0.6}, PDMS-MPU_{0.8}-IU_{0.2}, and PDMS-MPU_{0.6}-IU_{0.4}, the aspect ratio of the structures was better maintained, indicating higher structural stability. In addition, it was observed that the rate of change in pattern height (In ht, μm) relative to the initial height decreased over time.

Moreover, among the various MPU and IU molar ratios, PDMS-MPU_{0.8}-IU_{0.2} exhibited superior structural stability compared to PDMS-MPU_{0.4}-IU_{0.6} and PDMS-MPU_{0.6}-IU_{0.4}, making it more suitable for forming various types of structured geometries. In contrast, PDMS-MPU_{0.4}-IU_{0.6} and PDMS-MPU_{0.6}-IU_{0.4} showed faster reflow rates and relatively lower structural stability, making it difficult to retain the patterned structures.

### Experimental Example 2: Evaluation of Adhesion Properties of the Self-Healing Adhesive Patch

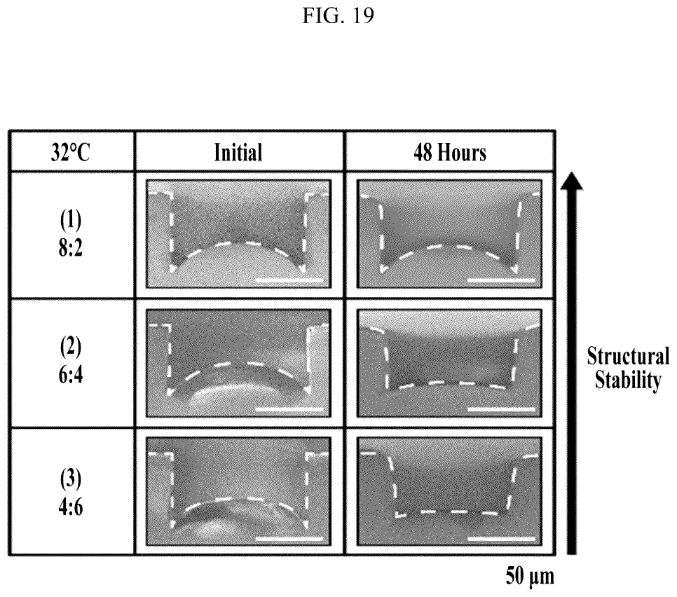

FIG. 19 shows optical microscope (OM) images comparing the structural stability of Example 1-1 according to the molar ratios of MPU and IU.

Referring to FIG. 19, Example 1-1 consists of three layers with different MPU and IU molar ratios: PDMS-MPU_{0.8}-IU_{0.2}, PDMS-MPU_{0.6}-IU_{0.4}, and PDMS-MPU_{0.4}-IU_{0.6}. OM analysis confirmed that the dome structures of PDMS-MPU_{0.6}-IU_{0.4} and PDMS-MPU_{0.4}-IU_{0.6} underwent significant deformation within 48 hours, whereas PDMS-MPU_{0.8}-IU_{0.2} maintained its structural integrity with minimal deformation even after 48 hours, indicating superior structural stability.

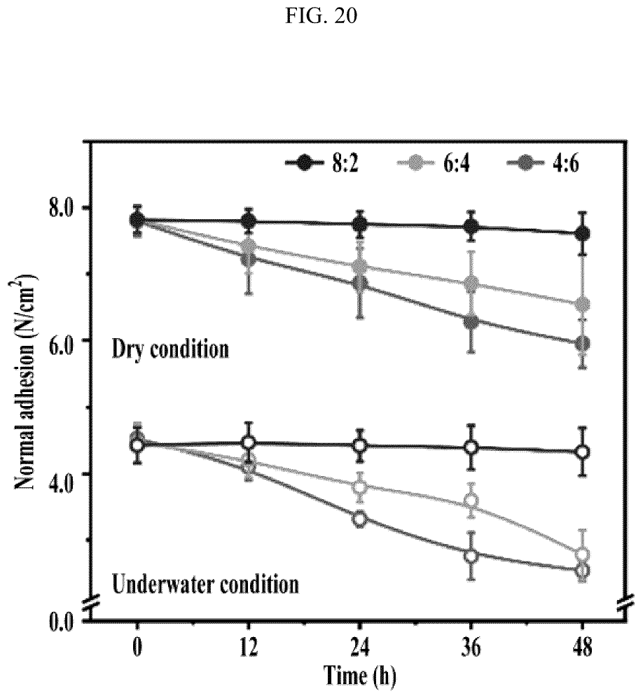

FIG. 20 is a graph evaluating the adhesion retention force of Example 1-1 according to the molar ratios of MPU and IU.

Referring to FIG. 20, the adhesive strength was measured on a silicon substrate under a preload of 3.5 N to assess adhesion performance under both dry and underwater conditions. It was confirmed that the adhesion strength was higher under dry conditions than underwater, and PDMS-MPU_{0.8}-IU_{0.2} exhibited higher adhesion strength than PDMS-MPU_{0.6}-IU_{0.4} and PDMS-MPU_{0.4}-IU_{0.6} under both environmental conditions.

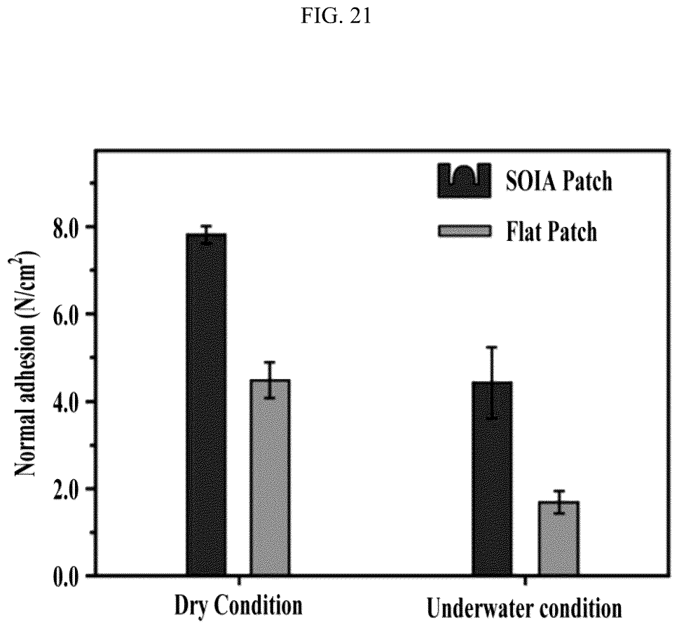

FIG. 21 is a graph comparing the adhesion performance of Example 1-1 and Comparative Example 1 under different environmental conditions.

Referring to FIG. 21, in dry conditions, Example 1-1 demonstrated a higher adhesion strength (7.82 N·cm⁻²) compared to Comparative Example 1 (4.485 N·cm⁻²), due to the effect of suction stress. In underwater conditions as well, Example 1-1 showed superior adhesion strength (4.43 N·cm⁻²) compared to Comparative Example 1 (1.69 N·cm⁻²).

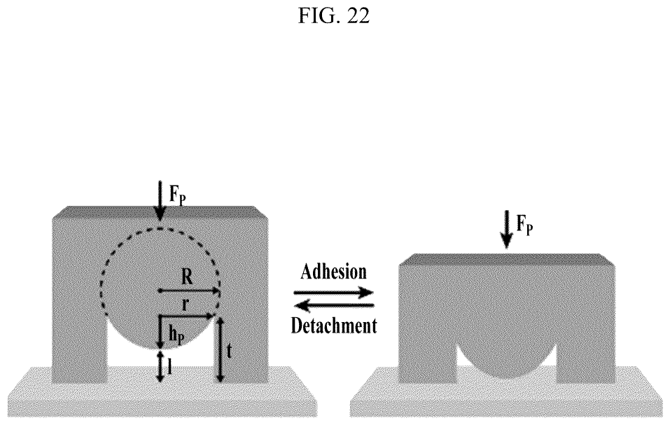

FIG. 22 shows images of the self-healing adhesive patch of Example 1 during attachment and detachment.

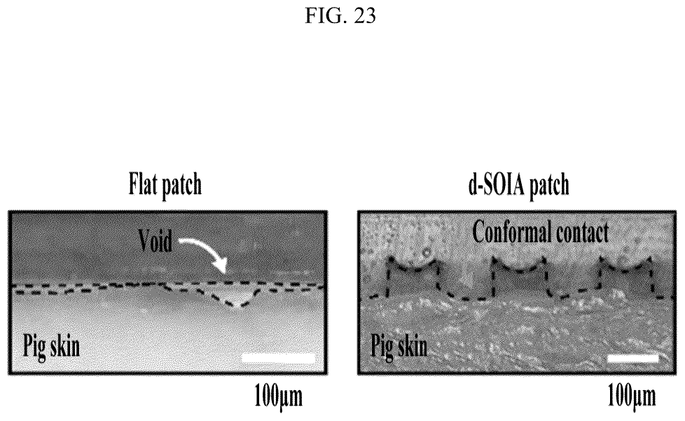

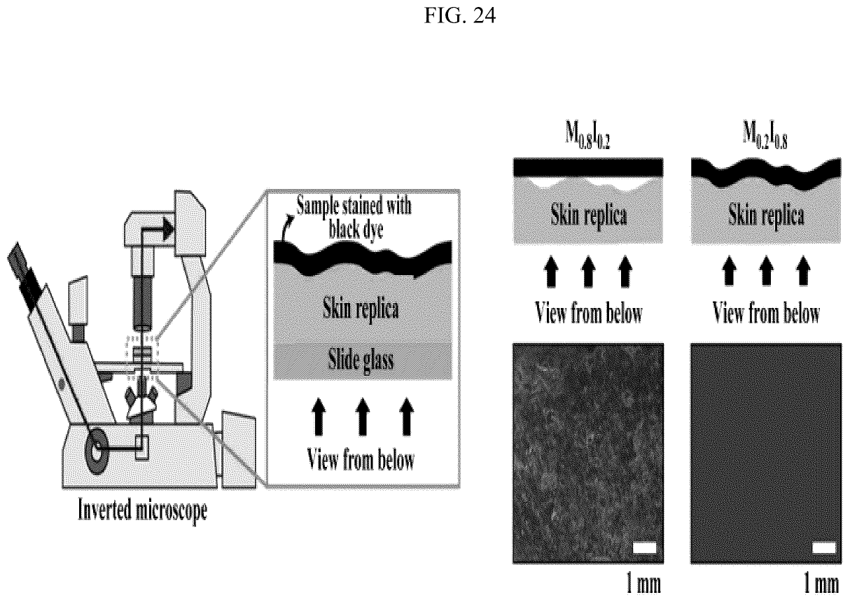

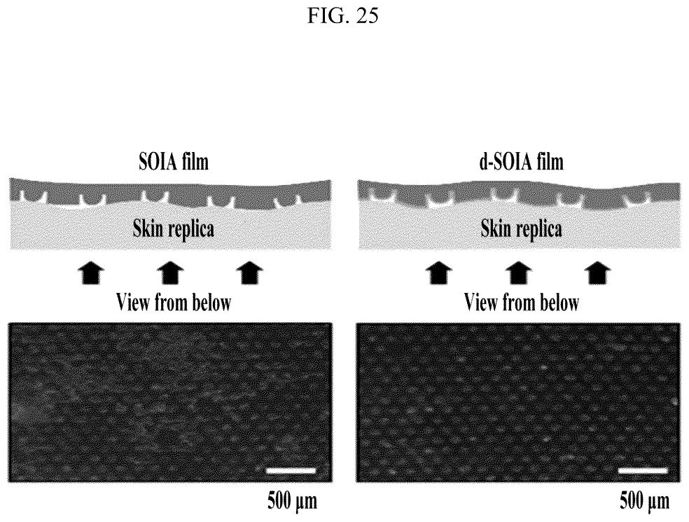

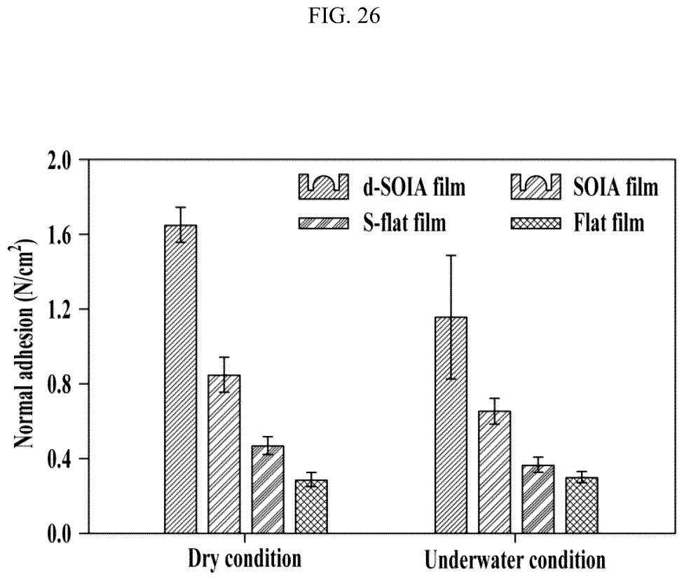

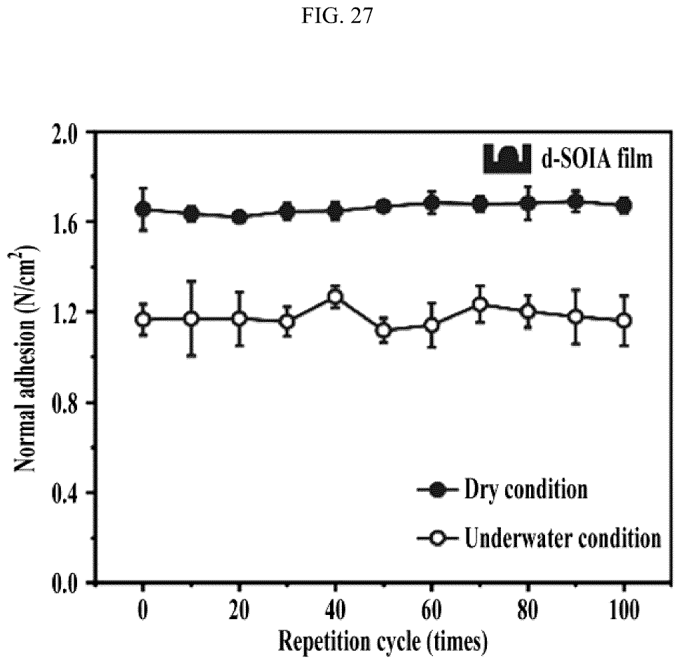

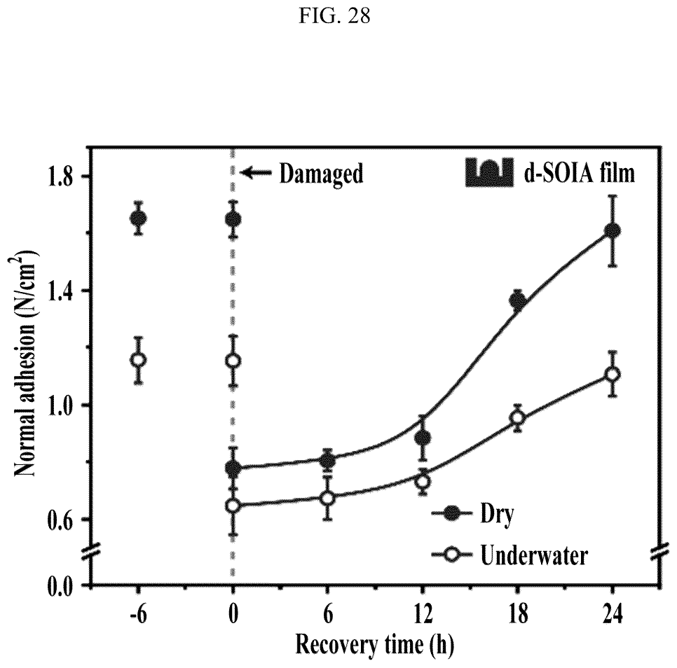

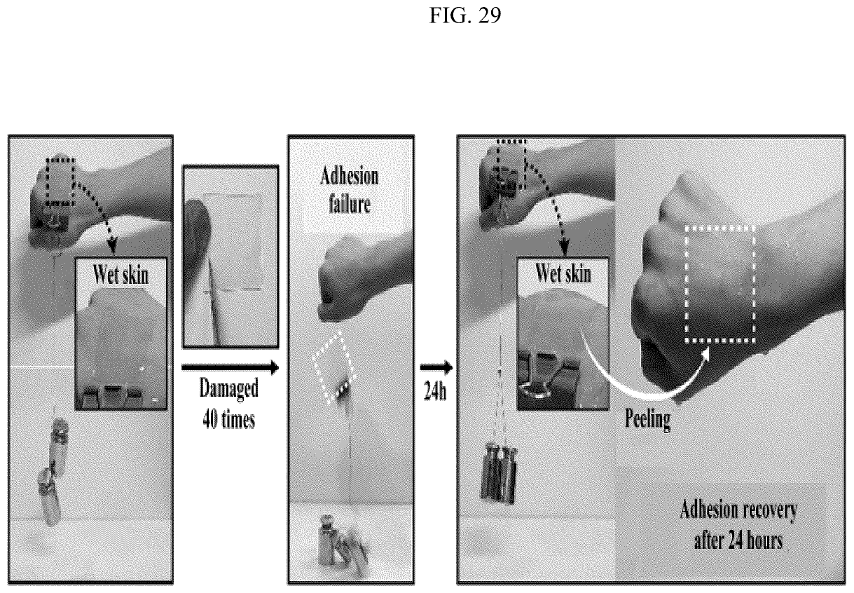

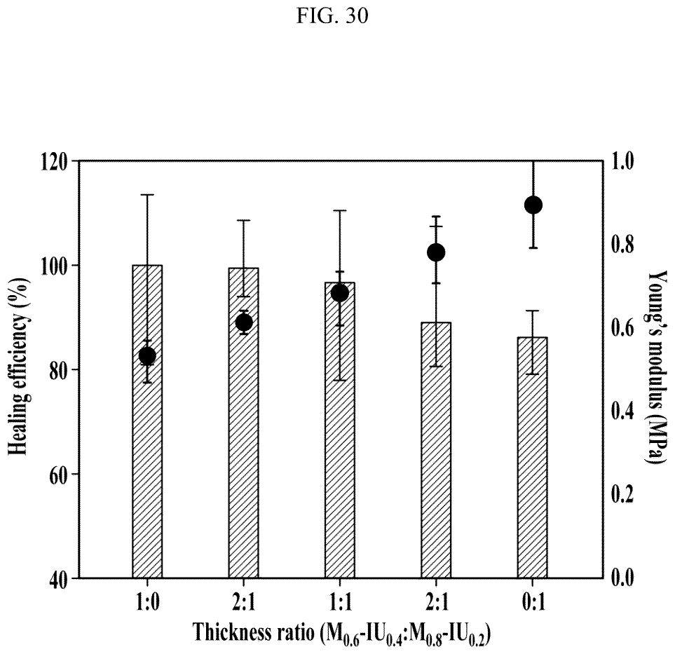

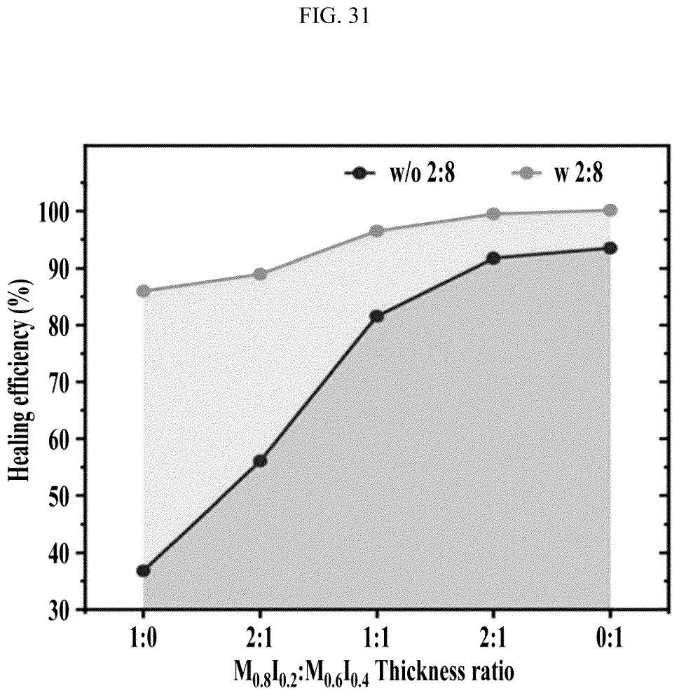

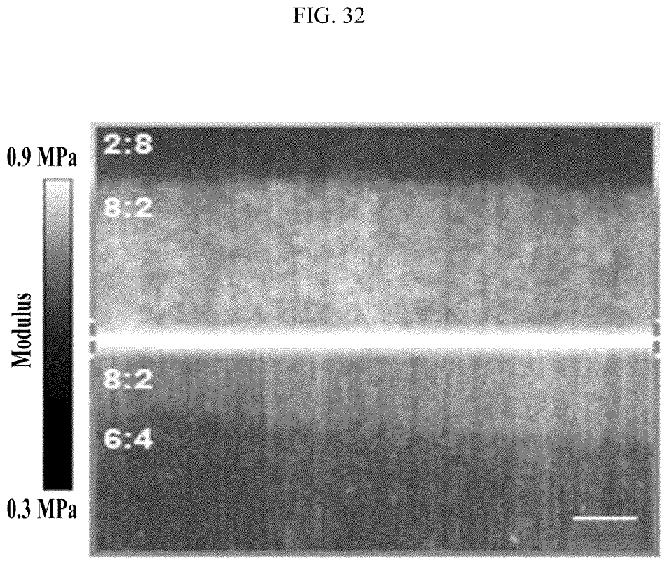

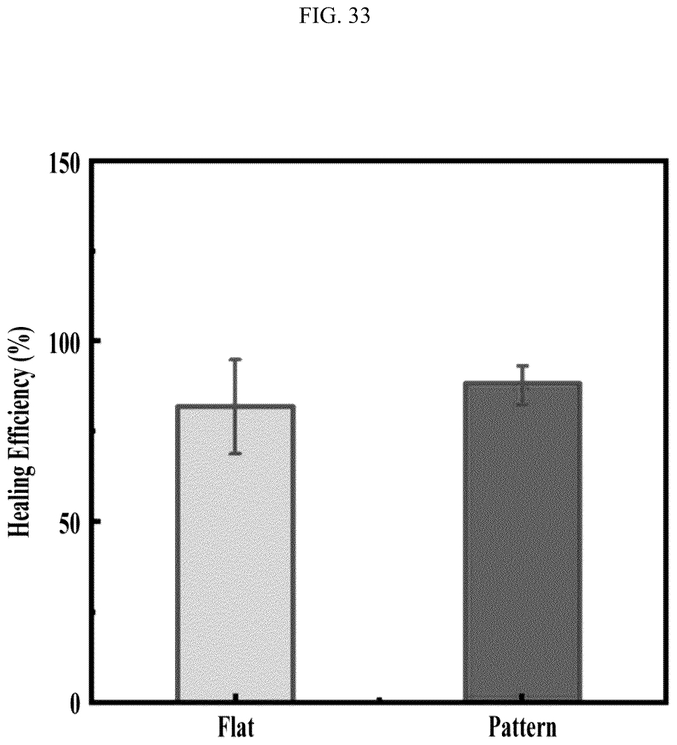

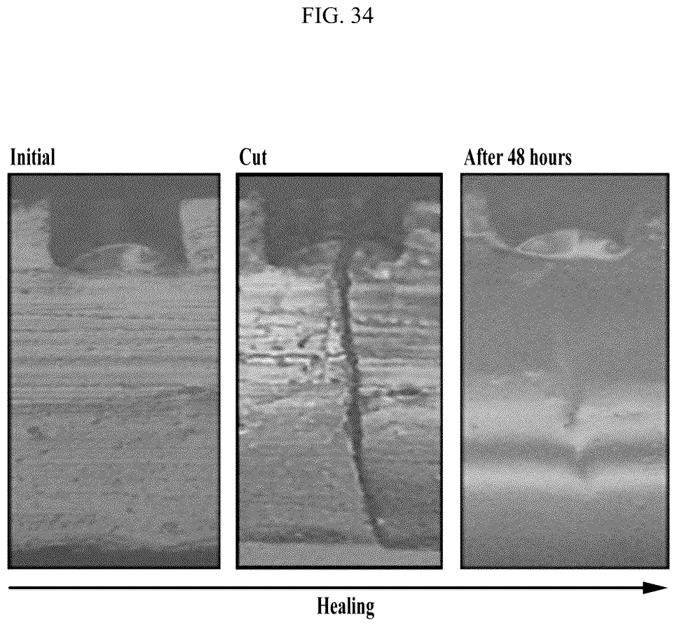

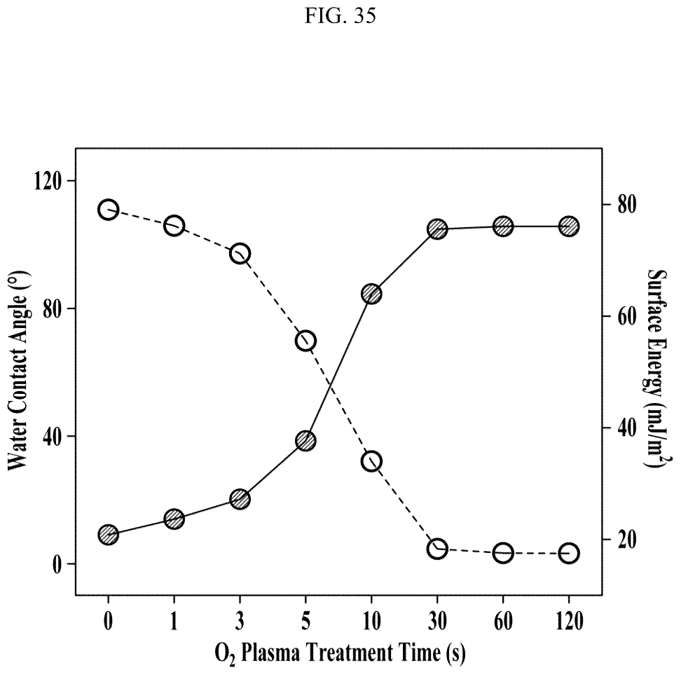

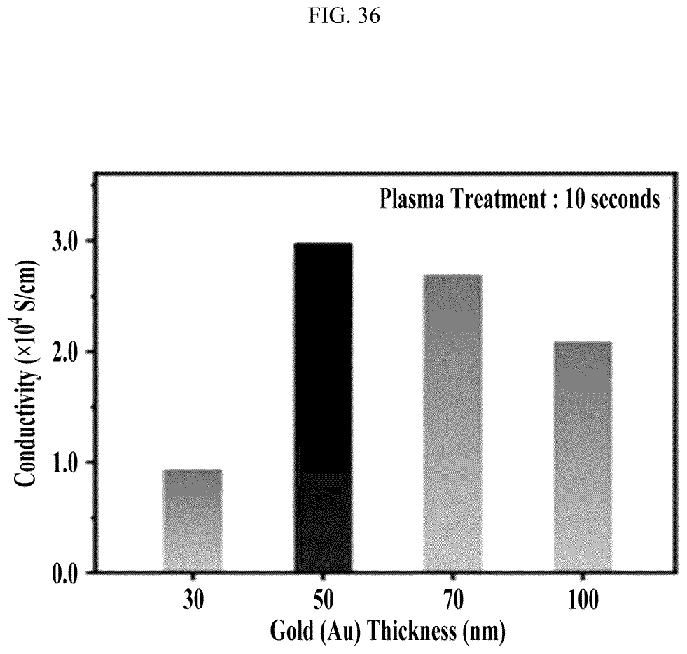

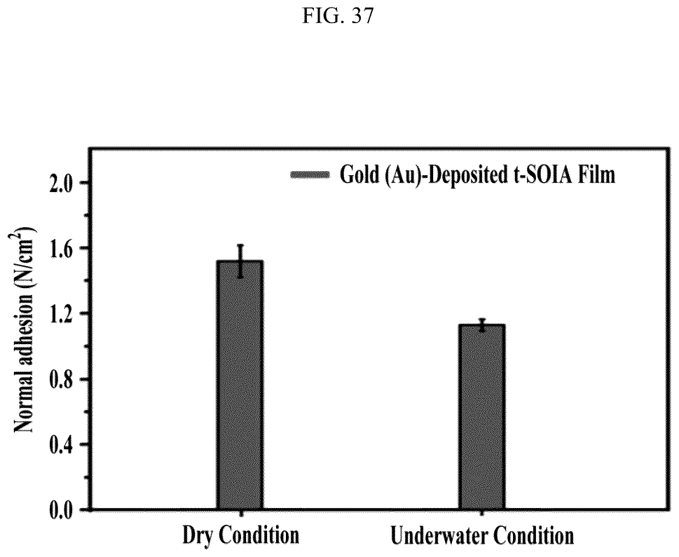

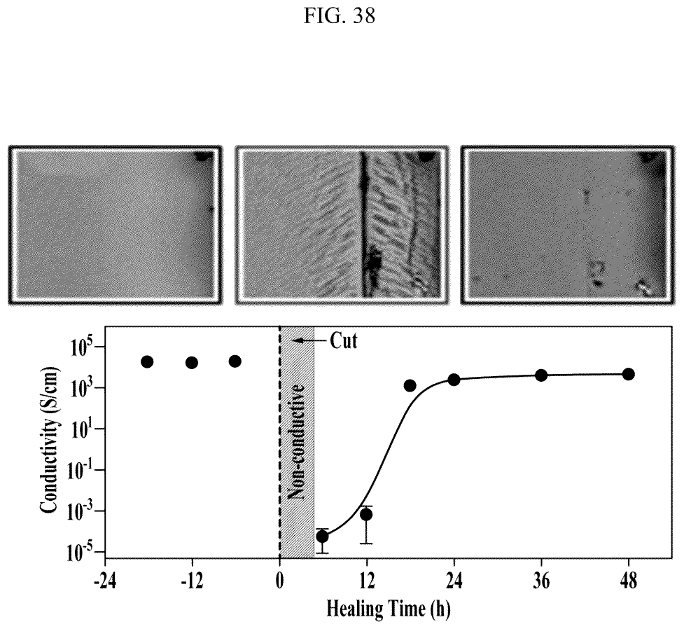

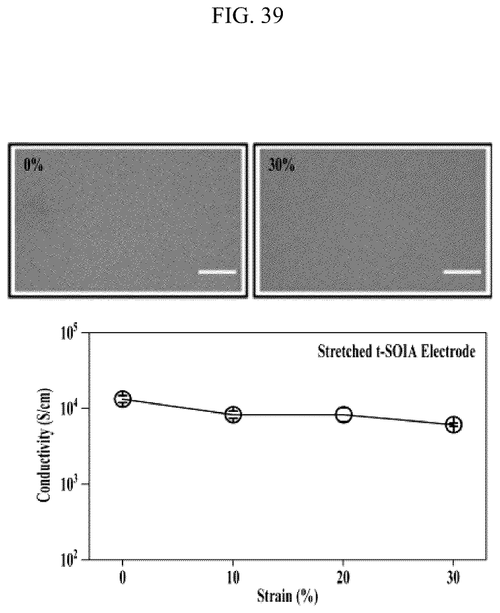

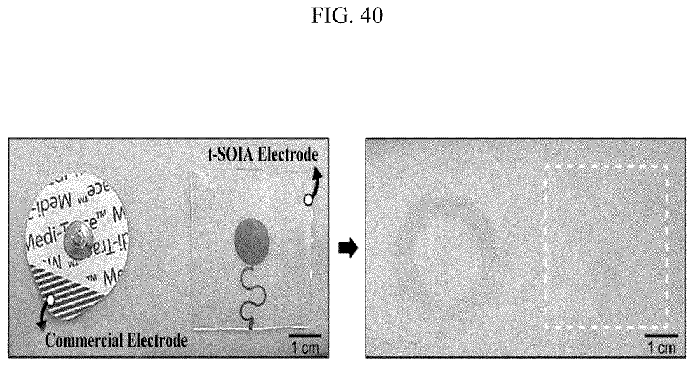

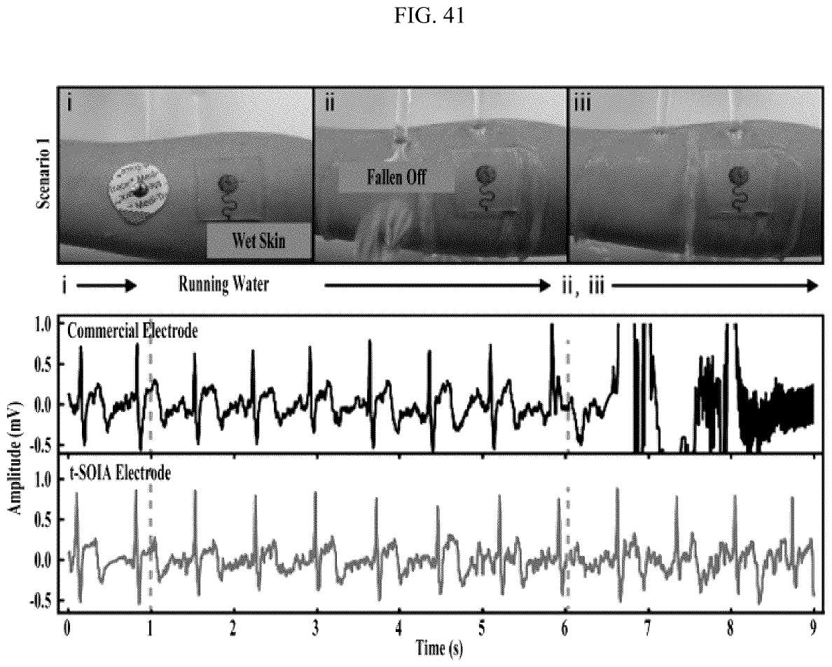

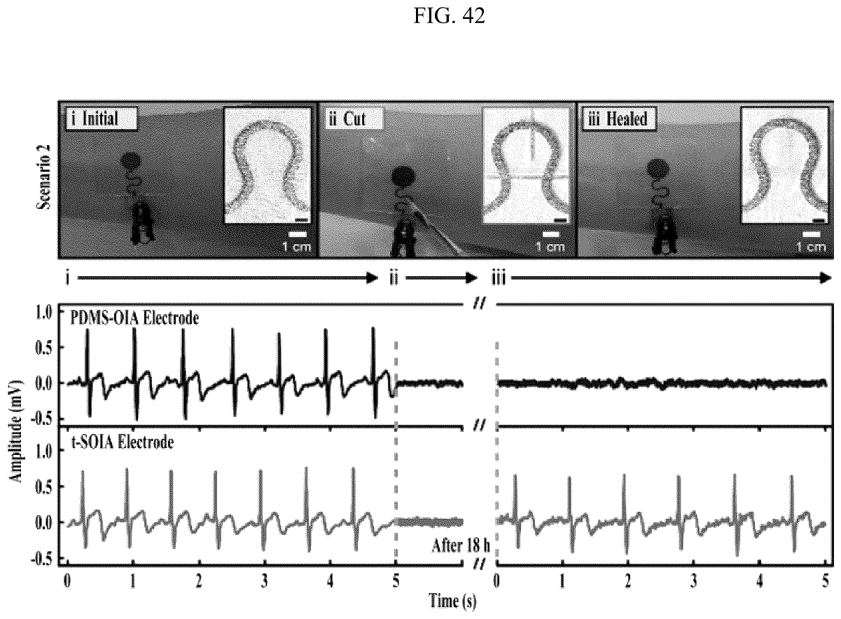

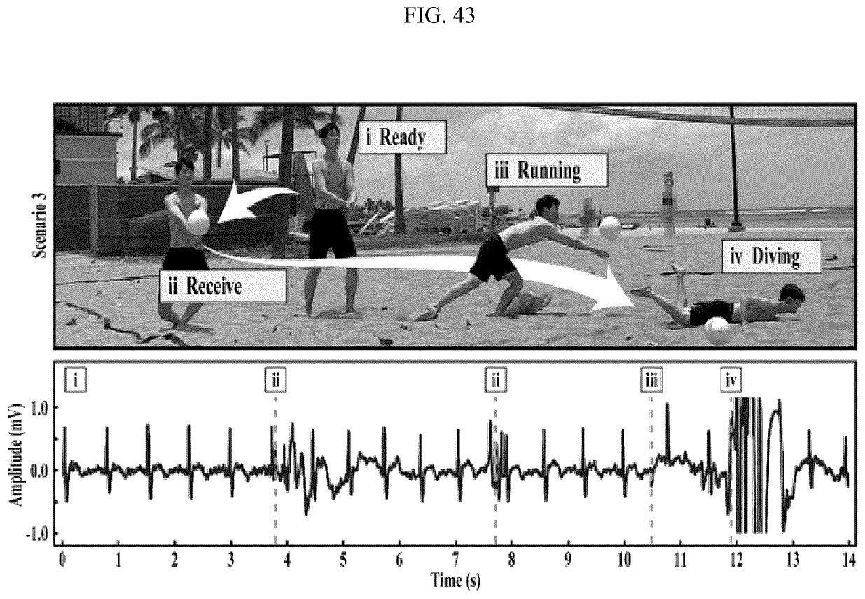

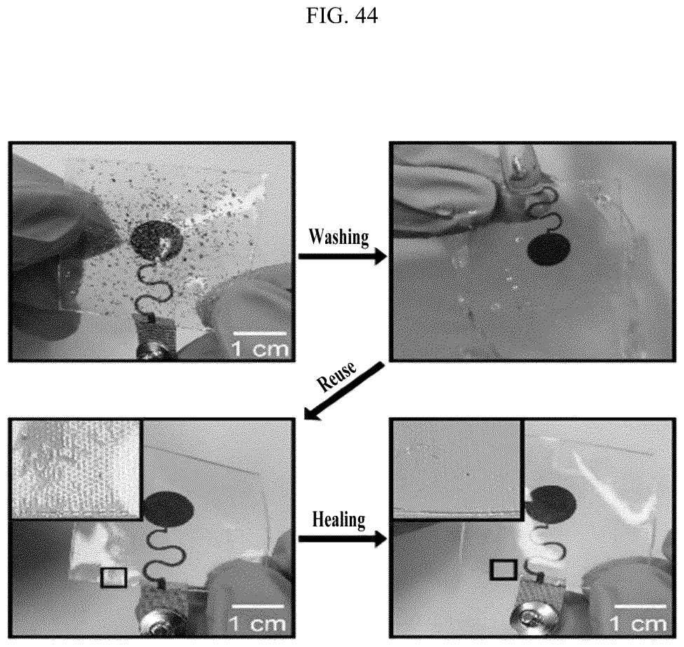

FIG. 23 shows comparative images of conformal contact between Example 1-2 and Comparative Example 1.

Referring to FIGS. 22 and 23, Example 1-2 employs soft and adaptive PDMS-MPU_{0.8}-IU_{0.2} to enhance surface contact efficiency on a pig skin substrate. The images confirm that the conformal contact achieved by the soft layer in Example 1-2 is superior to that of Comparative Example 1.

FIG. 24 illustrates a schematic diagram and OM images comparing conformal contact with and without PDMS-MPU_{0.2}-IU_{0.8}.

Referring to FIG. 24, the cross-sectional optical microscope images of adhesion on a pig skin substrate demonstrate that the conformal contact achieved by the soft layer in Example 1-2 is superior to that achieved by a flat layer without PDMS-MPU_{0.2}-IU_{0.8}.

FIG. 25 shows comparative optical microscope (OM) images of conformal contact for Example 1-1 and Example 1-2.

Referring to FIG. 25, it is confirmed that when PDMS-MPU_{0.2}-IU_{0.8} is applied as a soft layer on the top of Example 1-2, superior conformal contact is achieved compared to Example 1-1, which does not have the soft PDMS-MPU_{0.2}-IU_{0.8} layer. This improvement enhances the suction effect without leakage.

FIG. 26 is a graph evaluating the adhesion strength of various self-healing adhesive patches under different environmental conditions, and FIGS. 27 and 28 are graphs evaluating the repeated adhesion strength and resilience of Example 1-2.

Referring to FIGS. 26 through 28, the adhesion strength of Example 1-2 was higher than that of other patches under both dry and underwater conditions on pig skin. It was also confirmed that the repeated adhesion performance and self-healing resilience under repeated mechanical attachment-detachment cycles (>100) exhibited highly consistent behavior.

FIG. 29 shows images evaluating the self-adhesive healing capability of Example 1-2.

Referring to FIG. 29, to evaluate the self-adhesive healing capability of Example 1-2, surface-level cuts were introduced using a scalpel to mechanically damage the patterned layer, and the adhesion strength was monitored. It was observed that autonomous self-healing of the pattern and recovery of adhesion strength occurred after 24 hours without any external force. This result is attributed to the rapid self-healing characteristics of PDMS-MPU_{0.2}-IU_{0.8} applied in Example 1-2, which enables excellent adhesion retention even on rough and hairy skin surfaces.

### Experimental Example 3: Evaluation of Electrode Characteristics of the Self-Healing Adhesive Patch

FIGS. 30 and 31 are graphs illustrating the evaluation of self-healing performance and mechanical properties based on the thickness ratio of each layer in Examples 1-1 and 1-2.

Referring to FIG. 30, the optimal thickness ratio of the triple-layered structure in Example 1-1 was determined by considering healing efficiency and Young's modulus. When the thickness ratio of PDMS-MPU_{0.8}-IU_{0.2} and PDMS-MPU_{0.6}-IU_{0.4} was adjusted to 1:1, a healing efficiency of over 90% was achieved, indicating that the self-healing performance and mechanical properties were optimized.

Referring to FIG. 31, the thickness of the soft PDMS-MPU_{0.2}-IU_{0.8} in Example 1-2 was set to 20 μm, which is the minimum thickness required to accommodate the curvature of human skin. It was confirmed that the healing efficiency improved after applying PDMS-MPU_{0.2}-IU_{0.8} compared to before its application.

FIG. 32 is an image showing AFM analysis of the multilayered interface in Example 1-2.

Referring to FIG. 32, PeakForce Quantitative Nanomechanical Mapping (PeakForce QNM) analysis using an Atomic Force Microscope (AFM) was performed. Through the AFM analysis, it was confirmed that the optimized triple-layered interface composed of PDMS-MPU_{0.4}-IU_{0.6}, PDMS-MPU_{0.8}-IU_{0.2}, and PDMS-MPU_{0.2}-IU_{0.8} in Example 1-2 was clearly visualized.

FIG. 33 is a graph illustrating the evaluation of self-healing performance according to the presence or absence of a pattern in Example 2, and FIG. 34 is an optical microscope (OM) image showing the self-healing process of a fractured surface in Example 2.

Referring to FIGS. 33 and 34, the healing efficiency of the patterned patch in Example 2 was similar to that of Comparative Example 1. The optical microscope (OM) images confirmed that the scar of the cut patch in Example 2 was almost completely healed after 48 hours at 30° C.

FIG. 35 is a graph illustrating the evaluation of surface energy according to the oxygen (O₂) plasma treatment time in Example 3.

Referring to FIG. 35, prior to the deposition of gold (Au) in Example 3, the surface of the triple-layered film was treated with oxygen plasma. The surface energy was calculated using the Owens-Wendt method based on two test liquids: deionized water (DI-water) and diiodomethane. After 10 seconds of oxygen plasma treatment, the surface energy of the triple-layered film was saturated, indicating that a favorable interface with the Au electrode was achieved.

FIG. 36 is a graph illustrating the evaluation of electrical conductivity based on the thickness of the deposited gold (Au) in Example 3.

Referring to FIG. 36, the optimal electrical conductivity was observed when the thickness of the deposited Au was 50 nm.

FIG. 37 is a graph illustrating the evaluation of adhesion strength under various environmental conditions after Au deposition in Example 3.

Referring to FIG. 37, Example 3 exhibited excellent adhesion strength under both dry and underwater conditions, with particularly enhanced adhesion observed under dry conditions.

FIG. 38 is a graph illustrating the evaluation of electrical conductivity over self-healing time in Example 3, and FIG. 39 is a graph illustrating the evaluation of electrical conductivity under tensile deformation in Example 3.

Referring to FIG. 38, to evaluate the electrical self-healing capability of Example 3, the sample was completely cut using a scalpel, and the fractured surfaces were brought into contact again for 48 hours. It was confirmed that the electrical conductivity recovered to a value similar to the initial level (>10⁴ S/cm).

Referring to FIG. 39, Example 3 maintained stable conductivity even under tensile deformation up to 30%, without significant mechanical cracking or variation in resistance.

### Experimental Example 4: Evaluation of Electrocardiogram (ECG) Applicability of the Self-Healing Adhesive Patch

FIG. 40 is an image comparing skin irritation after attachment between Example 3 and Comparative Example 2.

Referring to FIG. 40, electrocardiogram (ECG) signals, one of the vital signs, were measured using Example 3, which included a gold (Au) electrode deposited and attached to human skin. To evaluate epidermal damage, both patches were attached and removed after the same period. It was confirmed that Example 3 achieved clean adhesion to the human skin surface, whereas Comparative Example 2 caused allergic reactions such as itching, stickiness, and redness on the attached skin area.

FIG. 41 is a graph comparing the performance of Example 3 and Comparative Example 2 under underwater conditions.

Referring to FIG. 41, Example 3 exhibited superior adhesion performance compared to Comparative Example 2 when applied to wet or rough skin, demonstrating high adhesive strength even under running water.

FIG. 42 is a graph evaluating the real-time self-healing process of Example 3, and FIG. 43 is a graph evaluating the real-time dynamic motion performance of Example 3.

Referring to FIG. 42, the ECG signal during the healing process reflected the disruption and recovery of conductive pathways. A stable signal was quickly restored, indicating the completion of the self-healing process. To verify real-time healing performance, PDMS-OIA and Example 3 were deliberately dissected using a scalpel. While PDMS-OIA did not recover to its original state even after sufficient time had passed, Example 3 fully restored the original signal after 18 hours.

Referring to FIG. 43, amplitude refers to the maximum displacement or distance moved from the center of oscillation during periodic vibration. The conformal contact characteristics of Example 3 were evaluated during beach volleyball activities (e.g., readying, running, receiving, diving), and it was confirmed that Example 3 maintained adhesion even during light and highly dynamic motion, enabling real-time tracking of heart rate and heart rate variability data.

FIG. 44 is an image evaluating the reusability of the self-healing adhesive patch according to Example 3.

Referring to FIG. 44, the reusability of Example 3 was evaluated. It was confirmed that Example 3 is reusable simply by rinsing off sea sand and dust on its hydrophobic surface, and that minor surface scratches caused by external sand can be effectively healed at room temperature due to its excellent self-healing performance.

Although the invention has been described with reference to the aforementioned embodiments and drawings, it is not limited thereto. Various modifications and alterations may be made by those skilled in the art without departing from the spirit and scope of the invention.

Therefore, the scope of the present invention should not be limited by the foregoing embodiments, but should be defined by the following claims and their equivalents.

What is claimed is:

1. A self-healing adhesive patch for an electronic skin sensor, comprising:
a first elastomer film layer comprising a polymer compound and an isocyanate-based compound;
a second elastomer film layer formed on the first elastomer film layer, the second elastomer film layer comprising the polymer compound and the isocyanate-based compound;
an electrode formed on the second elastomer film layer;
wherein the self-healing adhesive patch comprises the electrode on the second elastomer film layer;
wherein the isocyanate-based compound comprises a first isocyanate-based compound and a second isocyanate-based compound; and
wherein a first molar ratio of the first isocyanate-based compound to the second isocyanate-based compound in the first elastomer film layer is different from a second molar ratio of the first isocyanate-based compound to the second isocyanate-based compound in the second elastomer film layer.

2. The self-healing adhesive patch for an electronic skin sensor of claim 1,
wherein the first elastomer film layer and the second elastomer film layer are configured to control a Young's modulus of the first elastomer film layer and the second elastomer film layer by adjusting hydrogen bonding between the polymer compound and the isocyanate-based compound.

3. The self-healing adhesive patch for an electronic skin sensor of claim 1,
wherein the first molar ratio and the second molar ratio are each in a range of 0.1:0.9 to 0.9:0.1.

4. The self-healing adhesive patch for an electronic skin sensor of claim 1,
wherein a thickness ratio of the first elastomer film layer to the second elastomer film layer is in a range of 1:2 to 2:1.

5. The self-healing adhesive patch for an electronic skin sensor of claim 1,
wherein the second elastomer film layer comprises a pattern.

6. The self-healing adhesive patch for an electronic skin sensor of claim 1,
wherein the self-healing adhesive patch further comprises a third elastomer film layer formed on the second elastomer film layer;
wherein the third elastomer film layer comprises the polymer compound and the isocyanate-based compound;
wherein the isocyanate-based compound comprises the first isocyanate-based compound and the second isocyanate-based compound; and
wherein a third molar ratio of the first isocyanate-based compound to the second isocyanate-based compound in the third elastomer film layer is different from the first molar ratio and the second molar ratio.

7. The self-healing adhesive patch for an electronic skin sensor of claim 6,
wherein the third molar ratio is in a range of 0.1:0.9 to 0.9:0.1.

8. The self-healing adhesive patch for an electronic skin sensor of claim 6,
wherein a thickness of the third elastomer film layer is in a range of 20 μm to 30 μm.

9. The self-healing adhesive patch for an electronic skin sensor of claim 1,
wherein the polymer compound comprises at least one selected from the group consisting of polydimethylsiloxane (PDMS), polyethylene oxide (PEO), perfluoropolyether (PFPE), polybutylene (PB), poly (ethylene-co-1-butylene), poly (butadiene), hydrogenated poly (butadiene), poly (ethylene oxide)-poly (propylene oxide) block copolymer, and poly (hydroxyalkanoate).

10. The self-healing adhesive patch for an electronic skin sensor of claim 1,
wherein the isocyanate-based compound comprises at least one selected from the group consisting of 4,4′-methylenebis (phenyl isocyanate) (MPI), isophorone diisocyanate (IPDI), cyclohexylene diisocyanate, hydrogenated toluene diisocyanate (hydrogenated TDI), bis(2-isocyanatoethyl)-4-diclohexene-1,2-dicarboxylate, 2,5-norbornene diisocyanate, and 2,6-norbornene diisocyanate.

11. A method of manufacturing a self-healing adhesive patch for an electronic skin sensor, the method comprising:
forming a second elastomer film layer on a first elastomer film layer to produce an elastomer patch;
patterning the elastomer patch to produce an elastic adhesive patch;
performing plasma surface treatment on the elastic adhesive patch to produce a surface-modified elastic adhesive patch; and
depositing an electrode on the surface-modified elastic adhesive patch to produce the self-healing adhesive patch;
wherein the isocyanate-based compound comprises a first isocyanate-based compound and a second isocyanate-based compound; and
wherein a first molar ratio of the first isocyanate-based compound to the second isocyanate-based compound in the first elastomer film layer is different from a second molar ratio of the first isocyanate-based compound to the second isocyanate-based compound in the second elastomer film layer.

12. The method of manufacturing a self-healing adhesive patch for an electronic skin sensor of claim 11,
wherein, in the step of producing the elastomer patch, the first molar ratio and the second molar ratio are each in a range of 0.1:0.9 to 0.9:0.1.

13. The method of manufacturing a self-healing adhesive patch for an electronic skin sensor of claim 11,
wherein, in the step of producing the elastomer patch, the method further comprises forming a third elastomer film layer on the second elastomer film layer;
wherein the third elastomer film layer comprises the polymer compound and the isocyanate-based compound;
wherein the isocyanate-based compound comprises the first isocyanate-based compound and the second isocyanate-based compound; and
wherein a third molar ratio of the first isocyanate-based compound to the second isocyanate-based compound in the third elastomer film layer is different from the first molar ratio and the second molar ratio.

14. The method of manufacturing a self-healing adhesive patch for an electronic skin sensor of claim 11,
wherein, in the step of producing the elastomer patch, the third molar ratio is in a range of 0.1:0.9 to 0.9:0.1.

15. The method of manufacturing a self-healing adhesive patch for an electronic skin sensor of claim 11,
wherein, in the step of producing the elastic adhesive patch, the patterning is performed using one method selected from the group consisting of imprinting, 3D printing, and contact printing.
